(19) United States
(12) Patent Application Publication
(10) Pub. No.: US 2002/0115624 A1
(43) Pub. Date: Aug. 22, 2002

(54) Alpha gylcosylceramides for treating bacterial and fungal infections

(76) Inventors: Samuel M. Behar, Needham, MA (US); Michael B. Brenner, Newton, MA (US)

Correspondence Address: Elizabeth R. Plumer, Wolf, Greenfield & Sacks, P.C., Federal Reserve Plaza, 600 Atlantic Avenue, Boston, MA, 02210, US

(21) Appl. No.: 09/884,744

(22) Filed: Jun. 19, 2001

Related U.S. Application Data
(60) Provisional application No. 60/213,280, filed on Jun. 22, 2000.

(51) Int. Cl. A61K 31/7028; A61K 31/715

(52) U.S. Cl. 514/42; 514/54

(57) ABSTRACT

This invention relates to methods and compositions for the treatment of bacterial or fungal infectious disease, and to methods and compositions for screening assays to select agents that are useful for this purpose. In particular the invention relates to alpha-glycosylceramide molecules and their use in treating such infectious disease.

### RELATED APPLICATIONS

This application claims domestic priority under 35 U.S.C. §119(e) to U.S. Provisional Patent Application Serial No. 60/213,280, filed Jun. 22, 2000, incorporated herein in its entirety by reference.

### GOVERNMENT SUPPORT

This invention was made in part with government support under grant number HL64540 from the National Institutes of Health. The government may have certain rights in this invention.

### FIELD OF THE INVENTION

This invention relates to methods and compositions for the treatment of bacterial and fungal infections, and to methods and compositions for screening assays to select agents that are useful for this purpose. In particular, the invention relates to alpha-glycosylceramides, such as alpha-galactosylceramide and alpha-glucosylceramide, and their use in treating bacterial and fungal infections.

### BACKGROUND OF THE INVENTION

Alpha-galactosylceramide is one of a group of synthetic glycolipids that have been synthesized based on the structure of related compounds originally purified from marine sponges and shown to induce tumor regression in experimental animal models. Alpha-galactosylceramides are relatively nontoxic and are currently in human trials for the cancer immunotherapy. Accordingly, new therapeutic uses for alpha-glycosylceramides and like compounds would be expedited in view of the toxicity information presently available from human trials employing these compounds.

There have been literature reports that the recognition of alpha-glycosylceramides is a general feature of both human and murine NKT cells, a population of immunoregulatory T cells. Recognition reportedly is specific for the alpha-linkage (i.e., beta-galactosylceramide does not activate NKT cells) and certain sugars (galactose and glucose). The alpha-glycosylceramides are not known to be produced by mammalian cells or pathogenic microbes and their physiological relevance to the immune system is unknown.

Accordingly, a need exists to understand the relevance of the alpha-glycosylceramides to the mammalian immune system in order to develop new therapeutic uses for these compounds.

### SUMMARY OF THE INVENTION

We have discovered a new therapeutic use for alpha-glycosylceramides, namely, the treatment of bacterial and fungal infectious disease. Our discovery is based, in part, on the demonstration of a role for alpha-galactosylceramide in the treatment of murine tuberculosis. In view of this discovery and the availability of toxicity data from human trials employing these compounds for cancer immunotherapy, the transition into clinical trials of these compounds for the treatment of infectious disease should be facilitated.

Tuberculosis is among the infectious diseases that can be treated in accordance with the methods and compositions described herein. Worldwide, tuberculosis remains an important human pathogen. Except for AIDS, tuberculosis is responsible for more deaths than any other infectious disease. The global tuberculosis crisis has grown more severe due to the lack of new antibiotics and vaccines, the AIDS epidemic, and the emergence of multidrug resistant strains of M. tuberculosis. We have discovered, surprisingly, that administration of alpha-galactosylceramide dramatically and significantly prolongs the survival of mice infected with virulent M. tuberculosis. Although not wishing to be bound to any particular theory or mechanism, we believe that this effect of alpha-galactosylceramide, a known activator of CD1d restricted NKT cells, is mediated by modulating immunity to tuberculosis. Accordingly, we have investigated the role of this glycolipid in the treatment of tuberculosis and other bacterial or fungal infectious disease. We believe that the experiments described herein will lead to the development of improved therapy and compositions for treating such infectious disease.

As used herein, an NKT cell is a cell which is TCR (T cell receptor)-positive (and thus is considered a T lineage cell), but which also expresses various NK (natural killer) lineage markers. Although not wishing to be bound by any particular theory or mechanism, NKT cells are considered to be more closely related to the T cell lineage than the NK cell lineage. A significant fraction of CD1 restricted T cells are NKT cells, however, not all CD1 restricted T cells are NKT cells and not all NKT cells are CD1 restricted T cells. It is to be understood that as used herein, the term NKT cells is intended to embrace CD1 restricted NKT cells and that the methods and compositions of the invention which refer to NKT cells also intend to embrace CD1 restricted cells which may not be NKT lineage cells. Thus, the methods and compositions provided herein can be performed and made using non-NKT cell CD1 restricted cells.

According to one aspect of the invention, a method for treating infectious disease in a subject in need of such treatment is provided. The infectious disease is a bacterial infectious disease or a fungal infectious disease. The method involves administering to the subject, an alpha-glycosylceramide in an amount effective to treat the infectious disease in the subect. Preferably, the alpha-glycosylceramide is selected from the group consisting of an alpha-galactosylceramide and an alpha-glucosylceramide and the subject is not otherwise in need of administration of an alpha-galactosylceramide or an alpha-glucosylceramide. The preferred method of treatment further includes the step of detecting an improvement in the subject (e.g., reduction in bacterial burden on infected organs) following treatment.

Bacterial infectious diseases that can be treated in accordance with this method of the invention (administration by any route, preferably oral administration) include: Helicobacter pyloris, Borelia burgdorferi, Legionella pneumophilia, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus antracis, corynebacterium diphtheriae, corynebacterium sp., Erysipelothrix rhusiopathiae, Clostridium perfringers, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, Rickettsia, Actinomyces israelli, and Salmonella spp.

Fungal infectious diseases that can be treated in accordance with this method of the invention (administration by any route, preferably oral administration) include: Cryptococcus neoformans, Histoplasma capsulatum, Coccidioides immitis, Blastomyces dermatitidis, Chlamydia trachomatis, and Candida albicans.

The therapeutic methods of the invention involve administering to a subject an alpha-glycosylceramide. An alpha-glycosylceramide is a term of art which refers to class of naturally occurring or synthetic glycolipids that have been synthesized based on the structure of related compounds originally purified from marine sponges and shown to induce tumor regression in experimental animal models. Alpha-glycosylceramides have the general structural formula (A) depicted on page 3 in EP 0957161A1, entitled “Method for Activating Human Antigen Presenting Cells, Activated Human Antigen Presenting Cells, and Use of the Same”, Publication no. WO 98/29534, published Jul. 9, 1998 (referred to herein as “Kirin European Application”, incorporated in its entirety herein by reference). Exemplary alpha-glycosylceramides for use in accordance with the present invention include those depicted on pages 3 -10, inclusive, of the Kirin European Application, shown herein as Table 1 (following the Examples). In particular, this includes the compound referred to as KRN7000 (compound 14 in the Kirin European application table on page 8). Additional exemplary alpha-glycosylceramides for use in accordance with the present invention include those depicted in columns 1-15, inclusive, of the Kirin U.S. Pat. No. 5,936,076, entitled “alphaGalactosyl Derivatives”, issued Aug. 10, 1999 (referred to herein as “Kirin U.S. Pat. No. 5,936,076”, incorporated in its entirety herein by reference), also shown herein in Table 1.

An alpha-galactosylceramide is a term of art which refers to a molecule which has the general structure described above in which the carbohydrate moiety is galactose. Likewise, an alpha-glucosylceramide is a term of art which refers to a molecule which has general structure described above in which the carbohydrate moiety is glucose. Thus, as used herein, the alpha-glycosylceramides that are useful in accordance with the methods of the invention satisfy the conventional meaning of this phrase and are capable of treating an infectious bacterial or fungal disease as determined, for example, in animal models of the disease (See, e.g., the Examples). Alternatively, or additionally, alpha-glycosylceramides that are useful in accordance with the methods of the invention can be identified in screening assays which identify ceramides or functional analogs that are capable of stimulating (activating) NKT cells through a CD1d dependent mechanism. Screening assays for selecting such agents are described below.

According to another aspect of the invention, a pharmaceutical composition is provided. The composition includes: an alpha-glycosylceramide; an anti-infective agent that is an anti-bacterial agent or an anti-fungal agent; and a pharmaceutically acceptable carrier. In certain embodiments, the alpha-glycosylceramide is an alpha-galactosylceramide. In yet other embodiments, the alpha-glycosylceramide is an alpha-glucosylceramide. In these and other embodiments, the anti-infective agent is an anti-bacterial agent or, alternatively, an anti-fungal agent.

According to still another aspect of the invention, a screening method to identify putative alpha-glycosylceramide molecules that can stimulate NKT cells through a CD1d mechanism or that can be used to treat a bacterial or fungal infectious disease are provided. The method involves, in one embodiment, performing an NKT stimulation assay (e.g., cytokine release assay for cytokines such as but not limited to IL-2, IL-4, IL-10, IFN-γ, TGF-β and TNF-α) in the presence and absence of a putative alpha-glycosylceramide molecule; wherein an increase in the level of NKT cell stimulation in the presence of the putative ceramide molecule relative to the level of NKT cell stimulation in the absence of the putative ceramide molecule indicates that the putative ceramide molecule is an alpha-glycosylceramide as used herein. In particularly preferred embodiments, the NKT stimulation assay detects cytokine release profiles and, according to this embodiment, a shift in the Th1/Th2 profile. In one embodiment, the assay detects a shift towards a Th2 response in the presence of the putative agent to identify the agent as an alpha-glycosylceramide as used herein. In a preferred embodiment, the assay detects a shift toward a Th1 response in the presence of the putative agent to identify the agent as an alpha-glycosylceramide as used herein. In another important embodiment, the assay detects an alteration in NKT cell activation which may include assaying for the production and/or release of cytokines such as those listed above, or the upregulation, at either or both the transcriptional and translational level, of particular cell proteins (including, but not limited to, transcription factors, signal transduction factors and immune modulating factors), or the responsiveness of NKT cells to particular stimuli. In addition, assays which analyze similar activation parameters in cells other than NKT cells, including NK cells and T cells, are also embraced by the invention as useful assays for the identification of putative alpha-glycosylceramide agents.

These and other aspects of the invention, as well as various advantages and utilities, will be more apparent with reference to the detailed description of the preferred embodiments and to the accompanying drawings. Although the disclosure contains certain drawings, the drawings are not essential to the enablement of the claimed invention.

Certain terms used in this disclosure represent terms of art which have a meaning understood by one of ordinary skill in the art. Terms such as “effective amount” are defined in patents, such as those cited herein. Phrases such as “infectious disease”, “anti-infective agent”, “anti-bacterial agent”, and “anti-fungal agent” have well-established meanings to those of ordinary skill in the art and are defined in standard medical texts. Examples of particular ranges of effective amounts and infectious diseases are provided herein for illustrative purposes only and are not intended to limit the scope of the invention. Thus, it will be understood that various modifications may be made to the embodiments disclosed herein without departing from the essence of the invention. Therefore, the description of the invention should not be construed as limiting, but merely as exemplifications of preferred embodiments. Those skilled in the art will envision other modifications within the scope of the claims appended hereto.

All documents and publications identified herein are incorporated in their entirely herein by reference.

### BRIEF DESCRIPTION OF THE DRAWINGS

The Examples and Appendices include reference to one or more drawings that may or may not be present. It is to be understood that none of the drawings referenced in this application are required for enablement of the invention as disclosed herein.

FIG. 1. NKT cell interactions with DC can regulate the immune response. NKT cell recognition of alpha-GalCer, presented by CD1d⁺DC, can lead to the production of IL-12 by the DC. The IL-12 production is dependent on the CD40-CD40L interaction, and leads to the upregulation of IL-12R and IFN-gamma production by the NKT cell. The activation of NK cells by alpha-GalCer is dependent on NKT cells and their production of IFN-gamma. Under these circumstances, NKT cells may bias the immunity towards a Th1 response and inhibit a Th2 response.

FIG. 2. Left, Repro-ducibility of aerosol inoculation (Lung CFU one day after aerosol inoculation). Middle, Progression and dis-semination of infection following aerosol inoculation (Aerosol inoculation of BALB/c mice). Right, Survival following aerosol inoculation (Survival after aerosol infection).

FIG. 3. Intracellular cytokine staining of mononuclear cells isolated from the lung of an infected B6×129 F2 mouse six months after intravenous inoculation with M. tuberculosis. Cells were activated with PMA and ionomycin for 3.5 hours in the presence of brefeldin A (BFA), and then stained with anti-CD8 and fixed with 1% paraformaldehyde. Subsequently, the cells were permeabilized and stained for intracellular cytokines (in this case, IFN-gamma). Cells that had been cultured only with BFA, but not activated, did not show any significant IFN-gamma production. The numbers indicate the percentage of cells in each quadrant.

FIG. 4: Pulmonary T cell cytokine production after infection with M. tuberculosis. C57BL/6 or C3H/HeJ mice were infected and lung MNCs were prepared 1, 2, 3, or 4 weeks post infection, and analyzed to determine the number of cytokine producing cells. Cells were cultured with brefeldin A for 3.5 hrs with (activated: closed symbols) or without (unstimulated: open symbols) PMA & ionomycin. Five mice were pooled for each time point. Data is displayed as cells per ½ lung.

FIG. 5. Survival of CD1D and TAP1 knockout mice (broken lines) and the appropriate control mice (solid line). The genetic background of each pair is indicated in the parenthesis. 5A is CD1D −/− (C57BL/6); 5B is CD1D −/− (BALB/c); 5C is TAP1−/− (B6x 129). (See Behar, S. M., et al., 1999, Susceptibility of mice deficient in CD1D or TAP1 to infection with Mycobacterium tuberculosis, J. Exp. Med 189:1973-1980, for details).

FIG. 6. Survival of BALB/c mice infected with M. tuberculosis by the IV route and then treated with alpha-galactosylceramide (solid line) or vehicle alone (broken line). Nine mice were in each group and the survival analysis was done using the method of Kaplan and Meier. The difference in survival was statistically significant as determined by the log-rank test (p<0.0001).

### DETAILED DESCRIPTION OF THE INVENTION

The invention is based, in part, on the observation that administration of the glycolipid alpha-galactosylceramide prolonged the survival of mice infected with virulent Mycobacterium tuberculosis. Since we believe that the action of alpha-galactosylceramide in the treatment of infectious disease is mediated by CD1d-dependent NKT cells, the biology of CD1d and NKT cells is briefly reviewed below with a more detailed description of these components being provided in the Examples.

Group 1 (CD1a, b, and c) and group 2 (CD1d) CD1 proteins. The CD1 proteins are a family of antigen presenting molecules that, in contrast to the classical MHC class I and class II proteins, have evolved to present hydrophobic antigens to T cells. The human CD1 locus encodes a family of five nonpolymorphic proteins, CD1a-e, which are MHC class I-like. Analyses of the CD1 genes in humans and other species indicate that the proteins fall into two groups, CD1a, b, and c-like (group 1) and CD1d-like (group 2). The murine CD1 locus lacks the group 1 genes and contains only a duplicated group 2 gene. Murine CD1d is expressed by nearly all hematopoietic lineage cells, and at low levels by a variety of other cells.

It has become clear that the group 1 (CD1a, -b, and -c) proteins function to present foreign glycolipid antigens to diverse T cells, thereby significantly expanding the ability of the adaptive immune system to recognize and respond to pathogens. In contrast to these findings on CD1a, -b, and -c, work in both humans and mice indicates that CD1d interacts with a discrete population of immunoregulatory T cells. The identification of these immunoregulatory T cells in humans was initially based upon their unusual CD4⁻CD8⁻ phenotype and use of an invariant TCRalpha chain (Valpha24/JalphaQ without N-region diversity). Subsequent reports described the observation that murine NK1⁺ T cells used the homologous TCRalpha chain (Valpha14/Jalpha281) and recognized murine CD1d. Human invariant Valpha24-JalphaQ T cells are phenotypically and functionally homologous to murine NK¹⁺ T cells, and like their murine counterparts, are CD1d autoreactive, express NKR-P1 (CD161, the human homologue of NK1), and produce large amounts of IL-4 and IFN-gamma.

The literature reports have not supported a straightforward role for CD1d reactive NK1⁺ T cells in the development of an immune response. NKT cells are activated by IL-12 or more specifically by the synthetic glycolipid alpha-galactosylceramide. Administration of these agents in vivo leads to a rapid stimulation (activation) of NKT cells and induces a potent anti-tumor response that has been shown to significantly reduce the tumor burden in mice. While NKT cells have been shown to be both necessary and sufficient for an antitumor effect, administration of alpha-galactosylceramide to mice with intact immune systems leads to NKT cell dependent activation of multiple cell types including T cells, B cells, and macrophages.

Murine CD1d is recognized by a population of T cells that expresses NK1 (NKR-P1C), a cell surface C-type lectin, and use an invariant TCRalpha chain (Valpha14/Jalpha281) in association with Vbeta2, 7 or 8. NK1 is otherwise restricted to NK cells and these NK1⁺ T cells have been referred to as NKT cells or natural T cells. Phenotypically, NK1⁺ T cells are either CD4⁺CD8⁻ or CD4⁻CD8⁻ and this T cell population represents a major fraction of the mature T cells in thymus, a major T cell population in liver and up to 5% of splenic T cells.

Alpha-Glycosylceramides. The compound alpha-galactosylceramide is one of a group of synthetic glycolipids that have been synthesized based on the structure of related compounds originally purified from marine sponges and shown to induce tumor regression in experimental animal models. Taniguchi et al. have reported that the alpha-glycosylceramides are a class of glycolipid antigens presented by murine CD1d and recognized by invariant NKT cells. Subsequently, several groups have reported that the recognition of alpha-glycosylceramides is a general feature of both human and murine NKT cells. Recognition is specific for the alpha-linkage (i.e., beta-galactosylceramide does not activate NKT cells) and certain sugars (galactose and glucose). The presentation of α-glycosylceramides to NKT cells reportedly is TAP1-independent, but beta2-microglobulin and CD1d dependent. Although their structure resembles other CD1 presented antigens, the α-glycosylceramides are not known to be produced by mammalian cells or pathogenic microbes and their physiological relevance to the immune system is unknown.

It has been discovered that alpha-galactosylceramide has potent immunoregulatory effects when administered to mice in vivo. In general, all of these effects appear to be dependent upon CD1d restricted NKT cells, since activation of the immune system does not occur when alpha-galactosylceramide is administered to mice that lack CD1d (CD1d −/− mice) or lack NKT cells (Jalpha281−/− mice). Administration of alpha-galactosylceramide to mice leads to the rapid activation (within 3-24 hours) of NK, B, CD8⁺, and CD4⁺ lymphocytes, as determined by the induction of early cell activation markers such as CD69 (B, T, and NK cells) and CD80 and CD86 (B cells). In addition, following in vivo treatment with alpha-galactosylceramide, IFN-gamma production by NK cells occurs rapidly and an increase in serum IFN-gamma can be detected within 18 hours. Thus, we report herein our further discovery that the natural history of infectious bacterial disease can be modified by in vivo treatment with alpha-galactosylceramide or its functional analogs.

The foregoing observations and discoveries resulted in the inventions disclosed herein.

According to one aspect of the invention, a method for treating infectious disease in a subject in need of such treatment is provided. The infectious disease is a bacterial infectious disease or a fungal infectious disease. The method involves administering to the subject, an alpha-glycosylceramide in an amount effective to treat the infectious disease in the subect. Preferably, the alpha-glycosylceramide is selected from the group consisting of an alpha-galactosylceramide and an alpha-glucosylceramide and the subject is not otherwise in need of administration of an alpha-galactosylceramide or an alpha-glucosylceramide. The preferred method of treatment further includes the step of detecting an improvement in the subject (e.g., reduction in bacterial burden in affected organs) following treatment.

As used herein, the amount effective to treat the subject is that amount which inhibits either the development or the progression of an infectious disease or which decreases the rate of progression of an infectious disease. Thus, the treatment methods described herein also embrace prophylactic treatment of an infectious disease. The prophylactic method may further comprise, in another embodiment, the selection of a subject at risk of developing an infectious disease prior to the administration of the agent. Subjects at risk of developing an infectious disease include those who are likely to be exposed to an infectious agent. An example of such a subject is one who has been in contact with an infected subject, or one who is travelling or has traveled to a location in which a particular infectious disease in endemic. The prophylactic treatment methods provided may also include an initial step of identifying a subject at risk of developing an infectious disease. In some preferred embodiments, the prophylactic treatment may involve administering a vaccine to a subject.

As defined herein, an infectious disease or infectious disorder is a disease arising from the presence of a microbial agent in the body. The microbial agent may be an infectious bacteria or an infectious fungi, which gives rise to a bacterial infectious disease or a fungal infectious disease, respectively.

Examples of infectious bacteria (including mycobacteria) include but are not limited to: Helicobacter pyloris, Borelia burgdorferi, Legionella pneumophilia, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus antracis, corynebacterium diphtheriae, corynebacterium sp., Erysipelothrix rhusiopathiae, Clostridium perfringers, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, Rickettsia, Actinomyces israelli, and Salmonella spp.

Examples of infectious fungi include: Cryptococcus neoformans, Histoplasma capsulatum, Coccidioides immitis, Blastomyces dermatitidis, Chlamydia trachomatis, Candida albicans.

In one preferred embodiment, the microbial agent is one which causes a disease, the progression of which can be inhibited or halted by the presence of Th1 T cells and/or Th1 cytokines. Infectious diseases which can be favorably treated with Th1 cytokines include those caused by microbial agents, examples of which are salmonellosis and tuberculosis. In other important embodiments, the microbial agent is one which causes a disease, the progression of which can be inhibited or halted by the presence of Th2 T cells, Th2 cytokines, and more importantly, NK cells.

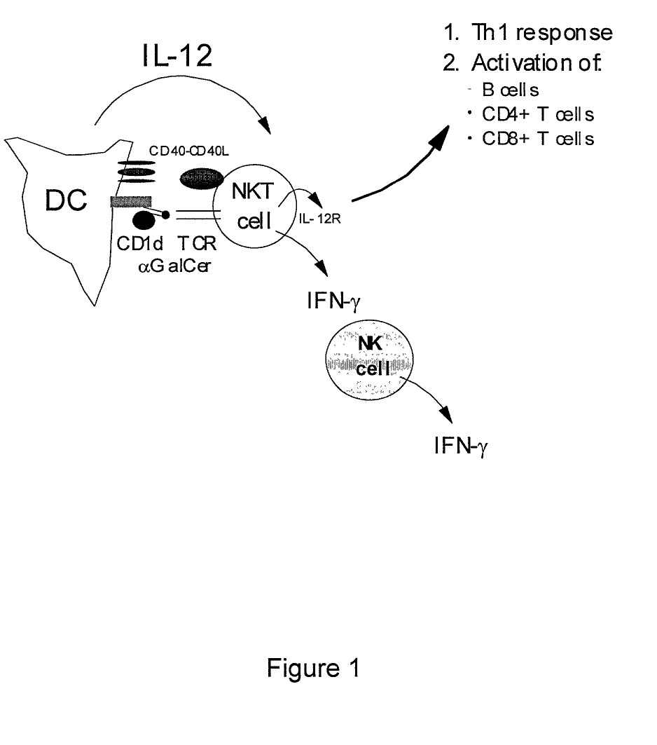

Although not wishing to be bound to any particular theory or mechanism, we believe that alpha-galactosylceramide stimulates NKT cells (as well as other CD1 restricted cells which may not be NKT lineage cells) through a CD1d mechanism. Accordingly, we believe that other ceramides or functional analogs which act through this mechanism also will be useful for treating infectious disease. Thus, the instant invention embraces other types of molecules, e.g., peptides, other small molecules such as those contained in molecular or chemical libraries, provided that such molecules are capable of stimulating NKT cells through a CD1d mechanism and, more preferably, which shift the Th1/Th2 balance in favor of a Th1 response. The Examples include a screening assay for detecting molecules which are capable of stimulating NKT cells through a CD1d mechanism as determined by measuring cytokine release profiles. (See, also, FIG. 1).

Putative alpha-glycosylceramide molecules which can be selected in screening assays and used in accordance with the present invention stimulate NKT cells through a CD1d mechanism as illustrated in the Examples. Thus, “stimulate NKT cells through a CD1d mechanism” refers to the ability of a putative alpha-glycosylceramide molecule to be presented by a CD1d molecule and, thereby, stimulate (i.e., activate) NKT cells resulting in, eg., cytokine release (such as, shifting to a Th1 release) by the cells. Accordingly, as used herein, an “alpha-glycosylceramide molecule” refers to a molecule (e.g., synthetic and naturally-occurring compounds) that: (1) is presented by CD1d and, thereby, (2) stimulates NKT cells and/or other CD1 restricted cells, as discussed earlier. The stimulation of NKT cells and/or other CD1 restricted cells in vitro is predictive of an in vivo effect. Accordingly, putative alpha-glycosylceramide molecules can be selected which favor a Th1 cytokine release profile and, thereby, enhance an immune response (e.g., to infection). In a similar fashion, putative alpha-glycosylceramide molecules can be selected which favor a Th2 cytokine release profile, or which stimulate NK cells, or which generally cause an alteration in the activation of NKT cells or other CD1 restricted cells.

The alpha-glycosylceramides of the invention are administered in effective amounts. An effective amount is a dosage of the alpha-glycosylceramide(s) sufficient to provide a medically desirable result. In general, a therapeutically effective amount means that amount necessary to delay the onset of, inhibit the progression of, or halt altogether the particular condition being treated. A therapeutically effective amount typically varies from 0.01 mg/kg to about 1000 mg/kg, preferably from about 0.1 mg/kg to about 200 mg/kg, and most preferably from about 0.2 mg//kg to about 20 mg/kg, in one or more dose administrations daily, for one or more days. Optimum dosages can be determined in accordance with standard procedures known to one of ordinary skill in the art. (See, e.g., the Examples.).

According to still another aspect of the invention, a screening method to identify putative alpha-glycosylceramide molecules that stimulate NKT cells in a CD1d mechanism for use in the therapeutic methods of the invention is provided. The method involves performing an NKT cell stimulation assay in the presence and absence of a putative alpha-glycosylceramide molecules; and determining the level of NKT cell stimulation in the presence and absence of the putative ceramide molecule, wherein an increase in the level of NKT cell stimulation in the presence of the putative ceramide molecule relative to the level of NKT cell stimulation in the absence of the putative ceramide molecule is an alpha-glycosylceramide molecule as used herein. In preferred embodiments, the NKT cell cytokine release profiles are obtained. In the most preferred embodiments, identification of an alpha-glycosylcermide of the invention is based on the detection of a shift in favor of a Th1 response in the presence of the putative ceramide molecule.

In a general sense, the invention embraces screening various types of libraries to identify alpha-glycosylceramide molecules and functional analogs (also referred to herein as “ceramide analogs” or “ceramide derivatives”) that are useful for practicing the invention. Thus, the preceding and following discussion is directed to the identification of alpha-glycosylceramide molecules and functional analogs that stimulate NKT cells in a CD1d dependent manner for use in accordance with the therapeutic methods disclosed herein. Preferably, the ceramide molecules and analogs stimulate a shift to a Th1 response.

Phage display can be effective in identifying ceramide analogs useful according to the invention. Yeast two-hybrid screening methods also may be used to identify polypeptides that function as alpha-glycosylceramide molecules in accordance with the methods of the invention. Compounds and libraries can be so tested for these abilities using screening assays such as those described below.

Alpha-glycosylceramide molecules and ceramide analogs can be synthesized using recombinant or chemical library approaches. A vast array of putative alpha-glycosylceramide molecules and analogs can be generated from libraries of synthetic or natural compounds. Libraries of natural compounds in the form of bacterial, fungal, plant and animal extracts are available or can be readily produced. Natural and synthetically produced libraries and compounds can be readily modified through conventional chemical, physical, and biochemical means. Known alpha-glycosylceramide molecules may be subjected to directed or random chemical modifications such as acylation, alkylation, esterification, amidification, etc. to produce structural analogs of these alpha-glycosylceramide molecules which stimulate NKT cell through a CD1d mechamism.

The methods of the invention utilize library technology to identify small molecules including small peptides which stimulate NKT cell through a CD1d mechanism. One advantage of using libraries for alpha-glycosylceramide molecule and ceramide analog identification is the facile manipulation of millions of different putative candidates of small size in small reaction volumes (i.e., in synthesis and screening reactions). Another advantage of libraries is the ability to synthesize ceramide analogs which might not otherwise be attainable using naturally occurring sources.

Methods for preparing libraries of molecules are well known in the art and many libraries are commercially available. Libraries of interest in the invention include glycolipid libraries, peptide libraries, randomized oligonucleotide libraries, synthetic organic combinatorial libraries, and the like. Degenerate peptide libraries can be readily prepared in solution, in immobilized form as bacterial flagella peptide display libraries or as phage display libraries. Peptides can be selected from combinatorial libraries of peptides containing at least one amino acid. Libraries can be synthesized of peptoids and non-peptide synthetic moieties. Such libraries can further be synthesized which contain non-peptide synthetic moieties which are less subject to enzymatic degradation compared to their naturally-occurring counterparts. Libraries are also meant to include for example but are not limited to peptide on plasmid libraries, polysome libraries, aptamer libraries, synthetic peptide libraries, synthetic small molecule libraries and chemical libraries. The libraries can also comprise cyclic carbon or heterocyclic structure and/or aromatic or polyaromatic structures substituted with one or more of the above-identified functional groups.

Small molecule combinatorial libraries may also be generated. A combinatorial library of small organic compounds is a collection of closely related analogs that differ from each other in one or more points of diversity and are synthesized by organic techniques using multi-step processes. Combinatorial libraries include a vast number of small organic compounds. One type of combinatorial library is prepared by means of parallel synthesis methods to produce a compound array. A “compound array” as used herein is a collection of compounds identifiable by their spatial addresses in Cartesian coordinates and arranged such that each compound has a common molecular core and one or more variable structural diversity elements. The compounds in such a compound array are produced in parallel in separate reaction vessels, with each compound identified and tracked by its spatial address. Examples of parallel synthesis mixtures and parallel synthesis methods are provided in U.S. Ser. No. 08/177,497, filed Jan. 5, 1994 and its corresponding PCT published patent application WO95/18972, published Jul. 13, 1995 and U.S. Pat. No. 5,712,171 granted Jan. 27, 1998 and its corresponding PCT published patent application WO96/22529, which are hereby incorporated by reference.

In certain embodiments, the libraries may have at least one constraint imposed upon the displayed peptide sequence. A constraint includes, e.g., a positive or negative charge, hydrophobicity, hydrophilicity, a cleavable bond and the necessary residues surrounding that bond, and combinations thereof. In certain embodiments, more than one constraint is present in each of the peptide sequences of the library.

As used herein, the term “ceramide analog” refers to a molecule which shares a common structural feature with the molecule to which it is deemed to be an analog. A “functionally equivalent” ceramide analog is an analog which further shares a common functional activity with the molecule to which it is deemed an analog. A “functionally equivalent non-ceramide analog is a compound which shares a common functional activity with the molecule to which it is deemed an analog, but may or may not share a common structural feature. For example, such non-ceramide analogs can be identified from combinatorial chemistry libraries by identifying molecules which have the desired functional activity. Non-ceramide analogs also include compounds which contain carbohydrate and/or hydrophobic moieties that are coupled to one another with a bond that approximates the same geometric distance as a ceramide but which is less susceptible to protease cleavage.

As used herein, the term “functionally equivalent ceramide analog” or “functional ceramide analog” refers to a ceramide analog that is capable of stimulate NKT cell through a CD1d mechanism and, more preferably, which stimulates a shift in favor of a Th1 response. Functionally equivalent ceramide analogs of alpha-galactosylceramide are identified, for example, in in vitro cytokine release assays (see, e.g., the assay provided in the Examples) that measure the ability of the ceramide analog to modulate cytokine release by NKT cells. Such assays are predictive of the ability of a molecule to modulate cytokine release in vivo.

The invention further provides compositions containing an alpha-glycosylceramide molecule in combination with an anti-bacterial and/or anti-fungal agent for improved anti-bacterial and/or anti-fungal therapy. Exemplary anti-bacterial agents include isoniazid; amoxicillin; clarithromycin; amoxicillin/clarithromycin combination; metronidazole; tetracycline, or naphthyridine carboxylic acid antibacterial compounds, polymyxin; rifampins; natural penicillins, semi-synthetic penicillins, clavulanic acid, cephalolsporins, bacitracin, ampicillin, carbenicillin, oxacillin, azlocillin, mezlocillin, piperacillin, methicillin, dicloxacillin, nafcillin, cephalothin, cephapirin, cephalexin, cefamandole, cefaclor, cefazolin, cefuroxine, cefoxitin, cefotaxime, cefsulodin, cefetamet, cefixime, ceftriaxone, cefoperazone, ceftazidine, moxalactam, carbapenems, imipenems, monobactems, euztreonam, vancomycin, polymyxin, amphotericin B, nystatin, imidazoles, clotrimazole, miconazole, ketoconazole, itraconazole, fluconazole, rifampins, ethambutol, tetracyclines, chloramphenicol, macrolides, aminoglycosides, streptomycin, kanamycin, tobramycin, amikacin, gentamicin, tetracycline, minocycline, doxycycline, chlortetracycline, erythromycin, roxithromycin, clarithromycin, oleandomycin, azithromycin, chloramphenicol, quinolones, co-trimoxazole, norfloxacin, ciprofloxacin, enoxacin, nalidixic acid, temafloxacin, sulfonamides, gantrisin, and trimethoprim. Still other anti-bacterial agents useful in the invention include Acedapsone; Acetosulfone Sodium; Alamecin; Alexidine; Amdinocillin; Amdinocillin Pivoxil; Amicycline; Amifloxacin; Amifloxacin Mesylate; Amikacin; Amikacin Sulfate; Aminosalicylic acid; Aminosalicylate sodium; Amphomycin; Ampicillin Sodium; Apalcillin Sodium; Apramycin; Aspartocin; Astromicin Sulfate; Avilamycin; Avoparcin; Azithromycin; Azlocillin; Azlocillin Sodium; Bacampicillin Hydrochloride; Bacitracin; Bacitracin Methylene Disalicylate; Bacitracin Zinc; Bambermycins; Benzoylpas Calcium; Berythromycin; Betamicin Sulfate; Biapenem; Biniramycin; Biphenamine Hydrochloride; Bispyrithione Magsulfex; Butikacin; Butirosin Sulfate; Capreomycin Sulfate; Carbadox; Carbenicillin Disodium; Carbenicillin Indanyl Sodium; Carbenicillin Phenyl Sodium; Carbenicillin Potassium; Carumonam Sodium; Cefaclor; Cefadroxil; Cefamandole; Cefamandole Nafate; Cefamandole Sodium; Cefaparole; Cefatrizine; Cefazaflur Sodium; Cefazolin; Cefazolin Sodium; Cefbuperazone; Cefdinir; Cefepime; Cefepime Hydrochloride; Cefetecol; Cefixime; Cefmenoxime Hydrochloride; Cefmetazole; Cefmetazole Sodium; Cefonicid Monosodium; Cefonicid Sodium; Cefoperazone Sodium; Ceforanide; Cefotaxime Sodium; Cefotetan; Cefotetan Disodium; Cefotiam Hydrochloride; Cefoxitin; Cefoxitin Sodium; Cefpimizole; Cefpimizole Sodium; Cefpiramide; Cefpiramide Sodium; Cefpirome Sulfate; Cefpodoxime Proxetil; Cefprozil; Cefroxadine; Cefsulodin Sodium; Ceftazidime; Ceftibuten; Ceftizoxime Sodium; Ceftriaxone Sodium; Cefuroxime; Cefuroxime Axetil; Cefuroxime Pivoxetil; Cefuroxime Sodium; Cephacetrile Sodium; Cephalexin; Cephalexin Hydrochloride; Cephaloglycin; Cephaloridine; Cephalothin Sodium; Cephapirin Sodium; Cephradine; Cetocycline Hydrochloride; Cetophenicol; Chloramphenicol; Chloramphenicol Palmitate; Chloramphenicol Pantothenate Complex; Chloramphenicol Sodium Succinate; Chlorhexidine Phosphanilate; Chloroxylenol; Chlortetracycline Bisulfate; Chlortetracycline Hydrochloride; Cinoxacin; Ciprofloxacin; Ciprofloxacin Hydrochloride; Cirolemycin; Clarithromycin; Clinafloxacin Hydrochloride; Clindamycin; Clindamycin Hydrochloride; Clindamycin Palmitate Hydrochloride; Clindamycin Phosphate; Clofazimine; Cloxacillin Benzathine; Cloxacillin Sodium; Cloxyquin; Colistimethate Sodium; Colistin Sulfate; Coumermycin; Coumermycin Sodium; Cyclacillin; Cycloserine; Dalfopristin; Dapsone; Daptomycin; Demeclocycline; Demeclocycline Hydrochloride; Demecycline; Denofungin; Diaveridine; Dicloxacillin; Dicloxacillin Sodium; Dihydrostreptomycin Sulfate; Dipyrithione; Dirithromycin; Doxycycline; Doxycycline Calcium; Doxycycline Fosfatex; Doxycycline Hyclate; Droxacin Sodium; Enoxacin; Epicillin; Epitetracycline Hydrochloride; Erythromycin; Erythromycin Acistrate; Erythromycin Estolate; Erythromycin Ethylsuccinate; Erythromycin Gluceptate; Erythromycin Lactobionate; Erythromycin Propionate; Erythromycin Stearate; Ethambutol Hydrochloride; Ethionamide; Fleroxacin; Floxacillin; Fludalanine; Flumequine; Fosfomycin; Fosfomycin Tromethamine; Fumoxicillin; Furazolium Chloride; Furazolium Tartrate; Fusidate Sodium; Fusidic Acid; Gentamicin Sulfate; Gloximonam; Gramicidin; Haloprogin; Hetacillin; Hetacillin Potassium; Hexedine; Ibafloxacin; Imipenem; Isoconazole; Isepamicin; Isoniazid; Josamycin; Kanamycin Sulfate; Kitasamycin; Levofuraltadone; Levopropylcillin Potassium; Lexithromycin; Lincomycin; Lincomycin Hydrochloride; Lomefloxacin; Lomefloxacin Hydrochloride; Lomefloxacin Mesylate; Loracarbef; Mafenide; Meclocycline; Meclocycline Sulfosalicylate; Megalomicin Potassium Phosphate; Mequidox; Meropenem; Methacycline; Methacycline Hydrochloride; Methenamine; Methenamine Hippurate; Methenamine Mandelate; Methicillin Sodium; Metioprim; Metronidazole Hydrochloride; Metronidazole Phosphate; Mezlocillin; Mezlocillin Sodium; Minocycline; Minocycline Hydrochloride; Mirincamycin Hydrochloride; Monensin; Monensin Sodium; Nafcillin Sodium; Nalidixate Sodium; Nalidixic Acid; Natamycin; Nebramycin; Neomycin Palmitate; Neomycin Sulfate; Neomycin Undecylenate; Netilmicin Sulfate; Neutramycin; Nifuradene; Nifuraldezone; Nifuratel; Nifuratrone; Nifurdazil; Nifurimide; Nifurpirinol; Nifurquinazol; Nifurthiazole; Nitrocycline; Nitrofurantoin; Nitromide; Norfloxacin; Novobiocin Sodium; Ofloxacin; Ormetoprim; Oxacillin Sodium; Oximonam; Oximonam Sodium; Oxolinic Acid; Oxytetracycline; Oxytetracycline Calcium; Oxytetracycline Hydrochloride; Paldimycin; Parachlorophenol; Paulomycin; Pefloxacin; Pefloxacin Mesylate; Penamecillin; Penicillin G Benzathine; Penicillin G Potassium; Penicillin G Procaine; Penicillin G Sodium; Penicillin V; Penicillin V Benzathine; Penicillin V Hydrabamine; Penicillin V Potassium; Pentizidone Sodium; Phenyl Aminosalicylate; Piperacillin Sodium; Pirbenicillin Sodium; Piridicillin Sodium; Pirlimycin Hydrochloride; Pivampicillin Hydrochloride; Pivampicillin Pamoate; Pivampicillin Probenate; Polymyxin B Sulfate; Porfiromycin; Propikacin; Pyrazinamide; Pyrithione Zinc; Quindecamine Acetate; Quinupristin; Racephenicol; Ramoplanin; Ranimycin; Relomycin; Repromicin; Rifabutin; Rifametane; Rifamexil; Rifamide; Rifampin; Rifapentine; Rifaximin; Rolitetracycline; Rolitetracycline Nitrate; Rosaramicin; Rosaramicin Butyrate; Rosaramicin Propionate; Rosaramicin Sodium Phosphate; Rosaramicin Stearate; Rosoxacin; Roxarsone; Roxithromycin; Sancycline; Sanfetrinem Sodium; Sarmoxicillin; Sarpicillin; Scopafungin; Sisomicin; Sisomicin Sulfate; Sparfloxacin; Spectinomycin Hydrochloride; Spiramycin; Stallimycin Hydrochloride; Steffimycin; Streptomycin Sulfate; Streptonicozid; Sulfabenz; Sulfabenzamide; Sulfacetamide; Sulfacetamide Sodium; Sulfacytine; Sulfadiazine; Sulfadiazine Sodium; Sulfadoxine; Sulfalene; Sulfamerazine; Sulfameter; Sulfamethazine; Sulfamethizole; Sulfamethoxazole; Sulfamonomethoxine; Sulfamoxole; Sulfanilate Zinc; Sulfanitran; Sulfasalazine; Sulfasomizole; Sulfathiazole; Sulfazamet; Sulfisoxazole; Sulfisoxazole Acetyl; Sulfisoxazole Diolamine; Sulfomyxin; Sulopenem; Sultamicillin; Suncillin Sodium; Talampicillin Hydrochloride; Teicoplanin; Temafloxacin Hydrochloride; Temocillin; Tetracycline; Tetracycline Hydrochloride; Tetracycline Phosphate Complex; Tetroxoprim; Thiamphenicol; Thiphencillin Potassium; Ticarcillin Cresyl Sodium; Ticarcillin Disodium; Ticarcillin Monosodium; Ticlatone; Tiodonium Chloride; Tobramycin; Tobramycin Sulfate; Tosufloxacin; Trimethoprim; Trimethoprim Sulfate; Trisulfapyrimidines; Troleandomycin; Trospectomycin Sulfate; Tyrothricin; Vancomycin; Vancomycin Hydrochloride; Virginiamycin; and Zorbamycin.

Exemplary anti-fungal agents include imidazoles, FK 463, amphotericin B, BAY 38-9502, MK 991, pradimicin, UK 292, butenafine, chitinase and 501 cream, Acrisorcin; Ambruticin; Amorolfine, Amphotericin B; Azaconazole; Azaserine; Basifungin; Bifonazole; Biphenamine Hydrochloride; Bispyrithione Magsulfex; Butoconazole Nitrate; Calcium Undecylenate; Candicidin; Carbol-Fuchsin; Chlordantoin; Ciclopirox; Ciclopirox Olamine; Cilofungin; Cisconazole; Clotrimazole; Cuprimyxin; Denofungin; Dipyrithione; Doconazole; Econazole; Econazole Nitrate; Enilconazole; Ethonam Nitrate; Fenticonazole Nitrate; Filipin; Fluconazole; Flucytosine; Fungimycin; Griseofulvin; Hamycin; Isoconazole; Itraconazole; Kalafungin; Ketoconazole; Lomofungin; Lydimycin; Mepartricin; Miconazole; Miconazole Nitrate; Monensin; Monensin Sodium; Naftifine Hydrochloride; Neomycin Undecylenate; Nifuratel; Nifurmerone; Nitralamine Hydrochloride; Nystatin; Octanoic Acid; Orconazole Nitrate; Oxiconazole Nitrate; Oxifungin Hydrochloride; Parconazole Hydrochloride; Partricin; Potassium Iodide; Proclonol; Pyrithione Zinc; Pyrrolnitrin; Rutamycin; Sanguinarium Chloride; Saperconazole; Scopafungin; Selenium Sulfide; Sinefungin; Sulconazole Nitrate; Terbinafine; Terconazole; Thiram; Ticlatone; Tioconazole; Tolciclate; Tolindate; Tolnaftate; Triacetin; Triafungin; Undecylenic Acid; Viridofulvin; Zinc Undecylenate; and Zinoconazole Hydrochloride.

In important embodiments, the preferred anti-microbial (including both anti-bacterial and anti-fungal) agents include ethambutol, isoniazid; rifampin; pyrazinamide; streptomycin, aminoglycosides, amikacin, kanamycin, tobramycin, gentamicin, ciprofloxacin, clofazimine, cycloserine, dapsone, ethionamide, ofloxacin, rifabutin; para-aminosalicylic acid; rifametane; rifamexil; rifamide; rifapentine; rifaximin; azithromycin, chloramphenicol, erythromycin; imipenem; clarithromycin; vancomycin; spectinomycin hydrochloride; polymyxin, amphotericin B, nystatin, imidazoles, clotrimazole, miconazole, ketoconazole, itraconazole, fluconazole.

The pharmaceutical preparations, as described above, are administered in effective amounts. The effective amount will depend upon the mode of administration, the particular condition being treated and the desired outcome. It will also depend upon, as discussed above, the stage of the condition, the age and physical condition of the subject, the nature of concurrent therapy, if any, and like factors well known to the medical practitioner. For therapeutic applications, it is that amount sufficient to achieve a medically desirable result.

Generally, doses of active compounds of the present invention would be from about 0.01 mg/kg per day to 1000 mg/kg per day. It is expected that doses ranging from 50-500 mg/kg will be suitable. Although a variety of administration routes are available, oral delivery is preferred in some embodiments particularly given its convenience to the subject. In other embodiments, aerosol and intravenous delivery may also be preferred. The methods of the invention, generally speaking, may be practiced using any mode of administration that is medically acceptable, meaning any mode that produces effective levels of the active compounds without causing clinically unacceptable adverse effects. Such modes of administration include aerosol, oral, rectal, topical, nasal, interdermal, or parenteral routes. The term “parenteral” includes subcutaneous, intravenous, intramuscular, or infusion. Intravenous or intramuscular routes are not particularly suitable for long-term therapy and prophylaxis. They could, however, be preferred in emergency situations. Oral administration will be preferred for prophylactic treatment because of the convenience to the patient as well as the dosing schedule. In certain embodiments a desirable route of administration is by pulmonary aerosol. Techniques for preparing aerosol delivery systems containing therapeutic agents are well known to those of skill in the art. Generally, such systems should utilize components which will not significantly impair the biological properties of the therapeutic agents (see, for example, Sciarra and Cutie, “Aerosols,” in Remington's Pharmaceutical Sciences, 18th edition, 1990, pp 1694-1712; incorporated by reference). Those of skill in the art can readily determine the various parameters and conditions for producing aerosols without resort to undue experimentation.

Compositions suitable for oral administration may be presented as discrete units, such as capsules, tablets, lozenges, each containing a predetermined amount of the active agent. Other compositions include suspensions in aqueous liquids or non-aqueous liquids such as a syrup, elixir or an emulsion.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Lower doses will result from other forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of compounds.

The alpha-glycosylceramide molecules of the invention, optionally including an anti-bacterial or anti-fungal agent, may be combined with a pharmaceutically-acceptable carrier. The term “pharmaceutically-acceptable carrier” as used herein means one or more compatible solid or liquid filler, diluents or encapsulating substances which are suitable for administration into a human. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction which would substantially impair the desired pharmaceutical efficacy.

When administered, the pharmaceutical preparations of the invention are applied in pharmaceutically-acceptable amounts and in pharmaceutically-acceptably compositions. Such preparations may routinely contain salt, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

The therapeutic methods of the invention involve administering to a subject an alpha-glycosylceramide. An alpha-glycosylceramide is a term of art which refers to a class of naturally occurring or synthetic glycolipids that have been synthesized based on the structure of related compounds originally purified from marine sponges and shown to induce tumor regression in experimental animal models. Alpha-glycosylceramides have the general structural formula (A) depicted on page 3) in EP 0957161A1, entitled “Method for Activating Human Antigen Presenting Cells, Activated Human Antigen Presenting Cells, and Use of the Same”, Publication no. WO 98/29534, published Jul. 9, 1998 (referred to herein as “Kirin European Application”, incorporated in its entirety herein by reference), shown herein as Table 1 (following the Examples). Exemplary alpha-glycosylceramides for use in accordance with the present invention include those depicted on pages 3-10, inclusive, of the Kirin European Application, and are enclosed herein as Table 1. In particular, this includes the compound referred to as KRN7000 (compound 14 in the Kirin European Application table on page 8), also shown herein in Table 1. Additional exemplary alpha-glycosylceramides for use in accordance with the present invention include those depicted in columns 1-15, inclusive, of the Kirin U.S. Pat. No. 5,936,076, entitled “alphaGalactosyl Derivatives”, issued Aug. 10, 1999 (referred to herein as “Kirin U.S. Pat. No. 5,936,076”, incorporated in its entirety herein by reference), shown herein as Table 2 (following the Examples).

An alpha-galactosylceramide is a term of art which refers to a molecule which has the general structure described above in which the carbohydrate moiety is galactose. Likewise, an alpha-glucosylceramide is a term of art which refers to a molecule which has general structure described above in which the carbohydrate moiety is glucose. Thus, as used herein, the alpha-glycosylceramides that are useful in accordance with the methods of the invention satisfy the conventional meaning of this phrase and are capable of treating an infectious bacterial or fungal disease as determined, for example, in animal models of the disease (See, e.g., the Examples). Alternatively, or additionally, alpha-glycosylceramides that are useful in accordance with the methods of the invention can be identified in screening assays which identify ceramides or functional analogs that are capable of stimulating (activating) NKT cells through a CD1d dependent mechanism.

Exemplary alpha-glycosylceramide molecules are described in the cited patent documents and are incorporated in their entirety herein. The Examples also provide screening assays for selecting putative ceramide molecules which are capable of stimulating NKT cells through a CD1d mechanism, particularly by shifting the Th1/Th2 balance in favor of a Th1 response. It is to be understood that other assays are also useful as screening methods including as described herein assays which measure Th2 cell and cytokine shifts, NK cell activation or stimulation, and general activation of CD1 restricted cells, whether or not they are NKT lineage cells. There are a large number of compounds described in the art that have been obtained naturally or synthetically, which can be tested using the screening assays disclosed herein to identify the category of molecules useful for practicing the present invention. However, the alpha-glycosylceramide derivatives disclosed in U.S. Pat. No. 5,973,128, entitled “Glycolipid mimics and methods of use thereof”, issued Oct. 26, 1999 (derivatized to include a rigid moiety which comprises at least one carbocyclic or heterocyclic ring element) are expressly excluded from the methods and compositions disclosed herein.

The pharmaceutical compositions may conveniently be presented in unit dosage form and may be prepared by any of the methods well-known in the art of pharmacy. All methods include the step of bringing the alpha-glycosylceramide molecules into association with a carrier which constitutes one or more accessory ingredients. In general, the compositions are prepared by uniformly and intimately bringing the alpha-glycosylceramide molecules into association with a liquid carrier, a finely divided solid carrier, or both, and then, if necessary, shaping the product.

In general, the alpha-glycosylceramide molecules can be administered to the subject (any mammalian recipient) using the same modes of administration that currently are used for administration of other anti-bacterial or anti-fungal agents in humans. A subject, as used herein, refers to any mammal (preferably a human, and including a non-human primate, cow, horse, pig, sheep, goat, dog, cat or rodent) that has and/or that is susceptible to a bacterial or fungal infectious disease. Preferably the mammal is otherwise free of symptoms calling for glycosylceramide treatment. Reported conditions that have symptoms calling for treatment with an alpha-glycosylceramide molecule include viral infectious such as HIV. Certain subjects with a condition for which known alpha-glycosylceramide molecules are prescribed for purposes other than the treatment of a bacterial or fungal infectious disease are hereby expressly excluded from the methods of the invention. These include subjects for which alpha-glycosylceramide molecules are prescribed to: treat viral infections and protist infections (e.g., malaria).

Other delivery systems can include time-release, delayed release or sustained release delivery systems. Such systems can avoid repeated administrations of the alpha-glycosylceramide molecules described above, increasing convenience to the subject and the physician. Many types of release delivery systems are available and known to those of ordinary skill in the art. They include the above-described polymeric systems, as well as polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono- di- and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which the alpha-glycosylceramide molecules is contained in a form within a matrix such as those described in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

Use of a long-term sustained release implant may be particularly suitable for treatment of chronic infection. Long-term release, as used herein, means that the implant is constructed and arranged to delivery therapeutic levels of the active ingredient for at least 30 days, and preferably 60 days. Long-term sustained release implants are well-known to those of ordinary skill in the art and include some of the release systems described above.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Each of the documents identified herein is incorporated in its entirety herein by reference.

The invention will be more fully understood by reference to the following examples. These examples, however, are merely intended to illustrate the embodiments of the invention and are not to be construed to limit the scope of the invention.

### EXAMPLES

### Example I

### A. Introduction

Worldwide, tuberculosis remains an important human pathogen. Except for AIDS, tuberculosis is responsible for more deaths than any other infectious disease. The global tuberculosis crisis has grown more severe due to the lack of new antibiotics and vaccines, the AIDS epidemic, and the emergence of multidrug resistant strains of M. tuberculosis. We have discovered that administration of α-galactosylceramide dramatically and significantly prolongs the survival of mice infected with virulent M. tuberculosis. Although not wishing to be bound to any particular theory or mechanism, we believe that this effect of α-galactosylceramide, a known activator of CD1d restricted NKT cells, is mediated by modulating immunity to tuberculosis. Accordingly, we have investigated the role of this glycolipid in the treatment of tuberculosis. Each of the following parameters/mechanism can be studied and/or optimized in accordance with standard procedures known to those of ordinary skill in the art and/or disclosed herein: (1) conditions under which alpha-galactosylceramide ameliorates tuberculosis; (2) the mechanism by which this compound modifies the disease process; (3) variables including the route of infection and the timing of treatment initiation; (4) the mechanism by which alpha-galactosylceramide reduces bacterial burden in affected organs; (5) the mechanism by which treatment alters tissue inflammation and pathology; and (6) (since we envision alpha-galactosylceramide as an immunomodulatory agent to be used in combination with conventional antimycobacterial chemotherapy), whether alpha-galactosylceramide acts synergistically with antibiotics such as isoniazid (INH). We believe that the experiments described herein will lead to the development of improved therapy for human tuberculosis. Our beliefs are based, in part, on the results disclosed herein.

### B. Background and Significance

The invention is based, in part, on the observation that administration of the glycolipid alpha-galactosylceramide prolongs the survival of mice infected with virulent Mycobacterium tuberculosis. Since the action of α-galactosylceramide is mediated by CD1d-dependent NKT cells, the biology of CD1d and NKT cells is reviewed below, followed by a discussion of the in vivo effects of alpha-galactosylceramide. Abbreviations used in the text: −/−, genetically deficient (i.e., “knockout”); alpha-GalCer, alpha-galactosylceramide; AIDS, acquired immunodeficiency syndrome; APC, antigen presenting cell; beta2m, beta2 microglobulin; CFU, colony forming unit; DC, dendritic cell; FCS, fetal calf serum; HIV, human immunodeficiency virus; IFN-gamma, gamma- interferon; IL, interleukin; INH, isoniazid; MAb, monoclonal antibody; MDR, muti-drug resistant; MHC, major histocompatibility complex; MNC, mononuclear cells; MST, mean survival time; NKT, natural killer T cell (i.e., NK1.1⁺ CD1d restricted T cells); TCR, T cell receptor; Th, T helper; TNF-alpha, tumor necrosis factor-alpha. Reference numbers appearing in parentheses are identified in the Example 1 reference list (below).

Group 1 (CD1a, b, and c) and group 2 (CD1d) CD1 proteins. The CD1 proteins are a family of antigen presenting molecules that, in contrast to the classical MHC class I and class II proteins, have evolved to present hydrophobic antigens to T cells. The human CD1 locus encodes a family of five nonpolymorphic proteins, CD1a-e, which are MHC class I-like based upon their structure including beta2-microglobulin association (Martin, L. H. et al., Proc. Natl. Acad. Sci. U.S.A., 84:9189-9193 (1987); Aruffo, A. and Seed, B., J. Immunol., 143:1723-1730 (1989); Balk, S. P. et al., Proc. Natl. Acad. Sci. U.S.A., 86:252-256 (1989); Blumberg, R. S. et al., Immunol. Rev., 147:5-29 (1995); Porcelli, S. A., Adv. Immunol., 59:1-98 (1995)). However, their sequences are quite divergent from MHC class I, class II, and other described nonclassical MHC-like proteins. Analyses of the CD1 genes in humans and other species indicate that the proteins fall into two groups, CD1a, b, and c-like (group 1) and CD1d-like (group 2) (Balk, S. P. et al., Proc. Natl. Acad. Sci. U.S.A , 86:252-256 (1989); Calabi, F. et al., Eur. J. Immunol., 19:285-292 (1989)). The murine CD1 locus lacks the group 1 genes and contains only a duplicated group 2 gene (Bradbury, A. et al., EMBO J., 7:3081-3086 (1988); Balk, S. P. et al., J. Immunol., 146:768-774 (1991)). Murine CD1d is expressed by nearly all hematopoietic lineage cells, and at low levels by a variety of other cells such as hepatocytes (Bleicher, P. A. et al., Science, 250:679-682 (1990); Mosser, D. D. et al., Immunology, 73:298-303 (1991); Blumberg, R. S. et al., J. Immunol., 147:2518-2524 (1991); Amano, M. et al., J. Immunol., 161:1710-1717 (1998)). The crystal structure of murine CD1d shows a deep hydrophobic pocket consistent with the ability to bind lipid or other hydrophobic antigens (Zeng, Z. et al., Science, 277:339-345 (1997)).

The biology of CD1. It has become clear that the group 1 (CD1a, -b, and -c) proteins function to present foreign glycolipid antigens to diverse T cells, thereby significantly expanding the ability of the adaptive immune system to recognize and respond to pathogens (Beckman, E. M. et al., Nature, 372:691-694 (1994); Beckman, E. M. et al., J. Immunol., 157:2795-2803 (1996); Sieling, P. A. et al., “CD1-restricted T cell recognition of microbial lipoglycan antigens”, Science, 269:227-230 (1995); Moody, D. B. et al., Science, 278:283-286 (1997)). In contrast to these findings on CD1a, -b, and -c, work in both humans and mice indicates that CD1d interacts with a discrete population of immunoregulatory T cells. The identification of these immunoregulatory T cells in humans was initially based upon their unusual CD4⁻CD8⁻phenotype and use of an invariant TCRα chain (Vα24/JαQ without N-region diversity) (Porcelli, S. et al., J. Exp. Med., 178:1-16 (1993)). Bendelac subsequently made the critical observation that murine NK1⁺ T cells used the homologous TCRα chain (Vα14/Jα281) and recognized murine CD1d (Bendelac, A. et al., Science, 268:863-865 (1995)). A function for murine CD1d reactive NK1⁺ T cells in regulating Th2 responses was suggested by their identification as the major source of early IL-4 after stimulation in vivo with anti-CD3 (Zlotnik, A. et al., J. Immunol., 149:1211-1215 (1992); Leite-de-Moraes, M. C. et al., J. Immunol., 155:4544-4550 (1995); Yoshimoto, T. and Paul, W. E., J. Exp. Med., 179:1285-1295 (1994); Yoshimoto, T. et al., Science, 270:1845-1847 (1995); Arase, H. et al., J. Immunol., 151:546-555 (1993); Arase, H. et al., J. Exp. Med., 183:2391-2396 (1996)). The homologous human invariant T cell clones similarly produce IL-4 in response to stimulation by anti-CD3 or CD1d (Exley, M. et al., J. Exp. Med., 186:109-120 (1997)). However, studies using β2-microglobulin deficient or CD1d knockout mice (both which lack cell surface CD1d and fail to positively select CD1d restricted T cells), have not supported a straightforward direct role for CD1d reactive NK1⁺ T cells in the development of Th2 responses (Brown, D. R. et al., J. Exp. Med, 184:1295-1304 (1996); Zhang, Y. et al., J. Exp. Med., 184:1507-1512 (1996); von der Weid, T. et al., J. Immunol., 157:4421-4427 (1996); Smiley, S. T. et al., Science, 275:977-979 (1997); Chen, Y. H. et al., Immunity, 6:459-467 (1997); Mendiratta, S. K. et al., Immunity, 6:469-477 (1997)). Recent work indicates instead a critical role for CD1d reactive NK1⁺ T cells in preventing autoimmunity and generating tumor immunity. Human invariant Vα24-JαQ T cells are phenotypically and functionally homologous to murine NK1⁺ T cells, and like their murine counterparts, are CD1d autoreactive, express NKR-P1 (CD161, the human homologue of NK1), and produce large amounts of IL-4 and IFN-γ(Exley, M. et al., J. Exp. Med., 186:109-120 (1997)). Furthermore, NKT cells are activated by IL-12 or more specifically by the synthetic glycolipid α-galactosylceramide. Administration of these agents in vivo leads to a rapid activation of NKT cells and induces a potent anti-tumor response that has been shown to significantly reduce the tumor burden in mice. While NKT cells have been shown to be both necessary and sufficient for the antitumor effect, administration of α-galactosylceramide to mice with intact immune systems leads to NKT cell dependent activation of multiple cell types including T cells, B cells, and macrophages.

Recognition of CD1d by T cells. Murine CD1d is recognized by a population of T cells that expresses NK1 (NKR-P1C), a cell surface C-type lectin, and use an invariant TCRα chain (Vα14/Jα281) in association with Vβ2, 7 or 8 (Coles, M. C. and Raulet, D. H., J. Exp. Med., 180:395-399 (1994); Adachi, Y. et al., Proc. Natl. Acad. Sci. U.S.A. 92:1200-1204 (1995); Arase, H. et al., Proc. Natl. Acad. Sci. U.S.A., 89:6506-6510 (1992); Koseki, H. et al., Proc. Natl. Acad. Sci. U.S.A., 87:5248-5252 (1990); Lantz, O. and Bendelac, A., J. Exp. Med 180:1097-1106 (1994)). NK1 is otherwise restricted to NK cells and these NK1⁺ T cells have been referred to as NKT cells or natural T cells (MacDonald, H. R., J. Exp. Med., 182:633-638 (1995); Bix, M. and Locksley, R. M., J. Immunol., 155:1020-1022 (1995)). Phenotypically, NK1⁺ T cells are either CD4⁺CD8⁻ or CD4⁻CD8⁻ and this T cell population represents a major fraction of the mature T cells in thymus, a major T cell population in liver and up to 5% of splenic T cells (Lantz, O. and Bendelac, A., J. Exp. Med. 180:1097-1106 (1994); Bendelac, A. et al., Science, 263:1774-1778 (1994); Makino, Y. et al., Proc. Natl. Acad. Sci. U.S.A., 93:6516-6520 (1996); Makino, Y. et al., J. Exp. Med., 177:1399-1408 (1993); Ohteki, T. and MacDonald, H. R., J. Exp. Med., 180:699-704 (1994)).

A significant advance in understanding the biology of the group 1 CD1 proteins (CD1a, b, and c) was the finding that these proteins can present foreign microbial lipid antigens including several mycobacterial antigens (Beckman, E. M., et al., J. Immunol., 157:2795-2803 (1996); Sieling, P. A., et al. Science, 269:227-230 (1995); Moody, D. B. et al., Science, 278:283-286 (1997); Beckman, E. M. Brenner, M. B., Immunol. Today, 16:349-352 (1995)). We have shown that mycolic acid, lipoarabinomannan, and lipids from mycobacterial species could be presented to human T cells by CD1b and CD1c (Beckman, E. M. et al., Nature, 372:691-694 (1994); Beckman, E. M. et al., J. Immunol., 157:2795-2803 (1996); Prigozy, T. I. et al., Immunity, 6:187-197 (1997); Sugita, M. et al., Science, 273:349-352 (1996)). In contrast, the antigens presented by CD1d remain poorly characterized. Work in this laboratory and others has shown that T cells recognize CD1d in the absence of exogenously added antigen (Bendelac, A. et al., Science, 268:863-865 (1995); Behar, S. M., J. Immunol., 162:161-167 (1999); Cardell, S. et al., J. Exp. Med., 182:993-1004 (1995)). This type of direct CD1d recognition, or “autoreactivity”, is remarkably conserved between species. For example, human T cells recognize murine CD1d and murine T cells recognize human CD1d (Brossay, L. et al., J. Exp. Med., 188:1521-1528 (1998)). Work from our laboratory has demonstrated that these directly reactive CD1d restricted T cells are antigen dependent and are likely to be recognizing endogenous cellular lipid antigens (Gumperz, J. E. et al., Immunity, 12:211-221 (2000)). While the physiological endogenous antigens that are presented by CD1d by APC remain largely unidentified, we have shown that some of these T cells recognize phospholipids including phosphatidylinositol and phosphatidylethanolamine.

α-Glycosylceramides. The compound α-galactosylceramide is one of a group of synthetic glycolipids that were synthesized based on the structure of related compounds originally purified from marine sponges and shown to induce tumor regression in experimental animal models (Morita, M. et al., J. Med. Chem., 38:2176-2187 (1995)). Taniguchi et al. reported that the α-glycosylceramides are a class of glycolipid antigens presented by murine CD1d and recognized by invariant NKT cells (Kawano, T. et al., Science, 278(5343):1626-1629 (1997)). Subsequently, several groups have reported that the recognition of α-glycosylceramides is a general feature of both human and murine NKT cells (Brossay, L. et al., J. Exp. Med., 188:1521-1528 (1998); Kawano, T., et al., Int. Immunol, 11:881-887 (1999); Spada, F. M. et al., J. Exp. Med., 188:1529-1534 (1998)). Recognition is specific for the α-linkage (i.e., β-galactosylceramide does not activate NKT cells) and certain sugars (galactose and glucose). The presentation of α-glycosylceramides to NKT cells reportedly is TAP 1-independent, but β2-microglobulin and CD1d dependent (Kawano, T. et al., Science, 278(5343):1626-1629 (1997)). Although their structure resembles other CD1 presented antigens, the α-glycosylceramides are not known to be produced by mammalian cells or pathogenic microbes and their physiological relevance to the immune system is unknown (Moody, D. B. et al., Science, 278:283-286 (1997); Kawano, T. et al., Science, 278(5343):1626-1629 (1997); Spada, F. M. et al., J. Exp. Med., 188:1529-1534 (1998)).

α-Galactosylceramide has potent immunoregulatory effects when administered in vivo. The α-glycosylceramides have profound immunological effects when administered to mice in vivo. In general, all of these effects appear to be dependent upon CD1d restricted NKT cells, since activation of the immune system does not occur when α-galactosylceramide is administered to mice that lack CD1d (CD1d −/− mice) or lack NKT cells (Jα281−/− mice). Administration of α-galactosylceramide to mice leads to the rapid activation (within 3-24 hours) of NK, B, CD8⁺, and CD4⁺ lymphocytes, as determined by the induction of early cell activation markers such as CD69 (B, T, and NK cells) and CD80 and CD86 (B cells) (Carnaud, C. et al., J. Immunol., 163:4647-4650 (1999); Burdin, N. et al., Eur. J. Immunol., 29:2014-2025 (1999); Singh, N. et al., J. Immunol. I, 163:2373-2377 (1999)). In addition, following in vivo treatment with α-galactosylceramide, IFN-γ production by NK cells occurs rapidly and an increase in serum IFN-γ can be detected within 18 hours.

The important issue of how α-galactosylceramide modulates T cell immune responses has not been definitively resolved. There are reports that α-galactosylceramide can skew the immune response of both NKT and conventional antigen specific T cells towards a Th2 phenotype (Burdin, N. et al., Eur. J. Immunol., 29:2014-2025 (1999); Singh, N. et al., J. Immunol. I, 163:2373-2377 (1999)). However, several studies have reported that CD1d −/− mice have intact Th2 responses (Smiley, S. T. et al., Science, 275:977-979 (1997)); Chen, Y. H. et al., Immunity, 6:459-467 (1997); Mendiratta, S. K. et al., Immunity, 6:469-477 (1997) and a recent study has reported that the absence of NKT cells (e.g., in Jα281 −/− mice) did not impair Th2 immune responses invivo (Cui, J. et al., J. Exp. Med., 190:783-792 (1999)). Similarly, the use of an in vitro culture system reported that activated NKT cells could inhibit Th2 cell differentiation (Cui, J. et al., J. Exp. Med., 190:783-792 (1999)). NKT cells produce enormous amounts of IFN-γ and we propose that, under certain circumstances, α-galactosylceramide can bias an immune response towards Th1 phenotype. Certainly, antitumor responses, such as those stimulated by α-galactosylceramide, are classically thought to be Th1 mediated responses. We believe that this conflicting data may ultimately be explained by the role of antigen presenting cells in the activation of NKT cells and at least two reports have emphasized the importance of dendritic cells (DC) in this process (Tomura, M. et al., J. Immunol, 163:93-101 (1999); Kitamura, H. et al., J. Exp. Med., 189:1121-1128 (1999)). Subsets of DCs have been defined in both mice and humans, and appear to be critical in the regulation of Th1-Th2 lymphocyte differentiation. Accordingly, we believe that the interaction between the DC and the NKT cell may be critical in determining whether an immune response becomes biased towards Th1 or Th2. For example, NKT cell recognition of α-galactosylceramide presented by DC, not only results in activation of the NKT cells, but also leads to the IL-12 production by DC. The IL-12 further stimulates NKT cells to produce IFN-γ, which has been reported to be important in the activation of NK cells. Complex interactions and feedback regulatory networks may determine whether activation of NKT cells leads to a Th1 or Th2 immune response (see FIG. 1).

The natural history of disease can be modified by in vivo treatment with α-galactosylceramide. CD1d reactive invariant T cells comprise a major fraction of the T cells in murine liver and can be stimulated by IL-12 to become active cytotoxic T cells and protect against liver metastases in tumor models (Hashimoto, W. et al., J. Immunol., 154:4333-4340 (1995); Takahashi, M. et al., J. Immunol., 156:2436-2442 (1996); Seki, S. et al., Immunology, 92:561-566 (1997)). NKT cells have been reported to be necessary through the generation of Jα281 knockout mice. These mice have markedly diminished numbers of invariant NK1+ T cells and reportedly, cannot mediate IL-12 induced tumor rejection (Cui, J. et al., Science 278(5343):1623-1626 (1997)). In contrast, mice expressing a transgenic invariant TCR (Vα14/Vβ8.2) on the RAG −/− background (so that this single TCR was expressed by all mature T cells) were able to mediate IL-12 dependent rejection of tumors. CD1d restricted NKT cells are thought to be important in generating IL-12 dependent immune responses, because IL-12 receptors are expressed by invariant NK1⁺ T cells and the early IFN-γ response by splenocytes and hepatic MNC following IL-12 administration reportedly is lost in CD1d −/− mice (invariant NK1⁺ T cell deficient) (Kawamura, T. et al., J. Immunol., 160:16 (1998)).

The antitumor effect of α-galactosylceramide was initially thought to be mediated by NK cells, but experiments performed in Jα281 knockout mice and TCR transgenic mice reportedly demonstrated that α-galactosylceramide induced tumor regression is also dependent upon NKT cells (Nakagawa, R. et al., Oncol. Res., 10:561-568 (1998); Nakagawa, R. et al., Cancer Res., 58:1202-1207 (1998)). Thus, the antitumor effect of both IL-12 and α-GalCer is dependent on CD1d restricted NKT cells. Although the antitumor effect of IL-12 is entirely reproduced by α-galactosylceramide, it is not known how NKT cells induce tumor regression. Tumoricidal activity is generated, but its mechanism does not seem to require cognate interaction between the tumor and NKT cells, and may be mediated by an NK-like activity. It is conceivable that NKT cells activate NK cells to kill tumor cells (Camaud, C. et al., J. Immunol., 163:4647-4650 (1999)). However, another report has emphasized that the killing of some tumors is dependent on NKT cells and not NK cells (Smyth, M. J. et al., J. Exp. Med, 191(4):661-668 (Feb.21, 2000)). The activation of anti-tumor responses appears to be an important pharmacological property of α-galactosylceramide and could be useful in the treatment of cancer, since α-galactosylceramide appears to be less toxic than IL-12.

The role of NKT cells and CD1 in tuberculosis. Short peptide antigens are presented by class I and class II MHC to conventional T cells. In contrast, the antigens presented by both group I and group II CD1 to T cells are lipid or glycolipid molecules composed of two acyl chains and a polar head group. Human T cells restricted by CD1a,-b, and -c have been reported to recognize mycobacterial lipid and glycolipid antigens, including mycolic acid, lipoarabinomannan (LAM), and glucose monomycolate (Beckman, E. M. et al., Nature, 372:691-694 (1994); Beckman, E. M. et al., J. Immunol., 157:2795-2803 (1996); Sieling, P. A. et al., Science, 269:227-230 (1995); Moody, D. B. et al., Science, 278:283-286 (1 997)). Furthermore, these antigens are presented by myeloid cells infected with M. tuberculosis, using the CD1 antigen processing pathway. Therefore, CD1 restricted T cells should be able to recognize infected macrophages. Since CD1 restricted CD8⁺ T cells express granulysin and can kill intracellular M. tuberculosis in an antigen specific CD1 restricted manner, it may be likely that such T cells could participate in microbial immunity (Stenger, S. et al., Science, 282:121-125 (1998)).

In contrast to group 1 CD1 (e.g., CD1a, -b, & -c), neither human nor murine CD1d restricted T cells specific for mycobacterial antigens have been identified, and it is unknown whether CD1d restricted T cells play a role in immunity to M. tuberculosis in mice. Our results from experiments using CD1D −/− mice in a high dose intravenous inoculation model of tuberculosis indicate that such T cells are not absolutely required for a protective immune response (see below and (Behar, S. et al., J. Exp. Med., 189:1973-1980 (1999))). However, Szalay et al. reported that anti-CD1d mAb administered in vivo to mice inoculated with M. tuberculosis impaired early immunity (Szalay, G. et al., Microbes. Infect., 1:1153-1157 (1999)). Although interesting, this study is difficult to interpret since CD1d is expressed on a variety of murine cell types and it was not shown whether this effect was secondary to the blockade of antigen presentation to CD1d restricted T cells. Preliminary data from Andrea Cooper (presented at the NKT Cell and CD1 Workshop, San Diego, Calif., 1999) reported that CD1d −/− mice inoculated with M. tuberculosis via the aerosol route had higher bacterial burdens in their lungs than control mice.

Perhaps the most provocative finding is that “deproteinized” M. tuberculosis cell walls injected subcutaneously into mice induced granuloma formation. The infiltrating T cells nearly exclusively used the invariant TCR α chain (Vα14-Jα281) that is characteristic of NKT cells (Apostolou, I. et al., [published erratum appears in Proc. Natl. Acad. Sci. U.S.A., 96(13):7610], Proc. Natl. Acad. Sci. U.S.A., 96:5141-5146 (Jun. 22, 1999)). In fact, granuloma formation under these conditions was entirely dependent upon NKT cells and granulomas failed to form in Jα281−/− mice, which lack NKT cells. The critical M. tuberculosis cell wall constituent appeared to be phosphatidylinositolmannosides (PIMs), since such compounds alone could induce granulomas containing infiltrating NKT cells. This is particularly interesting since PIM has been shown to be recognized by human CD1 restricted T cells. The compounds that have been reported to activate NKT cells in vivo are α-galactosylceramide and glycosylphosphatidyinositol (GPI) anchored antigen from parasites such as Plasmodium falciparum (Schofield, L. et al., Science, 283:225-229 (1992)). The recruitment and localization of NKT cells to these lipid induced granulomas is quite remarkable.

### C. Results

### Experimental Approaches to the Development of Therapeutic Modalities for the Treatment of Tuberculosis Using a Mouse Model

The details of certain methodologies, including survival analysis, assessment of tissue mycobacterial burden using CFU determination from organ homogenates, histopathological analysis of infected tissue, and flow cytometry are described below and in our prior publications (e.g., Gumperz, J. E. et al, Immunity., 12:211-221 (2000); Behar, S. M. et al, J. Exp. Med., 189:1973-1980 (1999) and Chackerian et al. (Appendix C)). Below we describe aerosol inoculation of mice with M. tuberculosis, and the use of intracellular cytokine flow cytometry. Finally, we end this section with the observations that form the basis for this application—the finding that α-galactosylceramide prolongs the survival of mice infected with M. tuberculosis.

### Intravenous Inoculation Model of Tuberculosis

In general, mice were inoculated with a high dose (10⁵-10⁶ cfu/mouse) via the lateral tail vein. Our laboratory uses the Erdman strain of M. tuberculosis, which we originally obtained from Dr. Barry Bloom (Harvard School of Public Health). We have maintained the strain's virulence by passaging it in mice, and limiting in vitro growth to two passages.

### Aerosol Inoculation Model of Tuberculosis

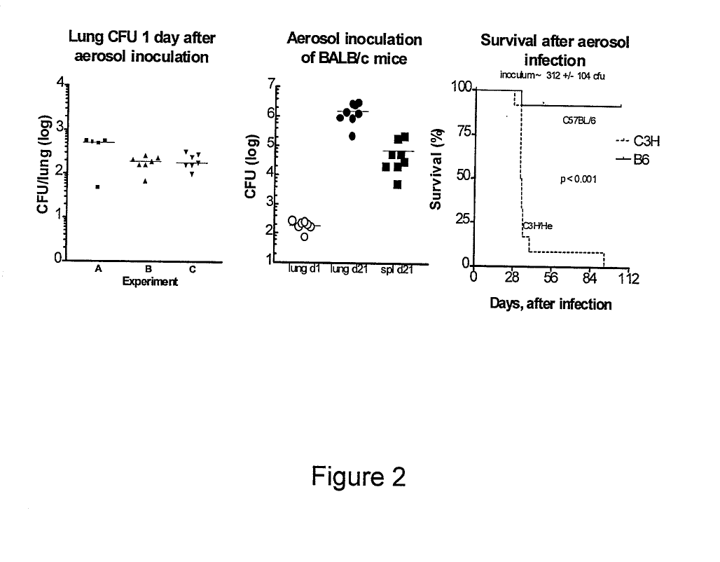

Although intravenous inoculation is a well established and widely used route of infection in the murine model of tuberculosis, inoculation by the aerosol route more closely mimics the natural mode of transmission between persons. Our laboratory uses a nose only aerosol delivery system, which provides the capability to deliver a small bacterial inoculum by the aerosol route into the lungs of mice and guinea pigs (see methods for further information). Other investigators using the aerosol inoculation route have used an inoculum size of between 50-500 cfu (Cooper, A. M. et al., J. Exp. Med., 178:2243-2247 (1993); Kelly, B. P. et al., Antimicrob. Agents Chemother., 40:2809-2812 (1996); North, R. J., Clin. Exp. Immunol. 113:55-58 (1998)). Preliminary experiments in this laboratory have been completed to calibrate our delivery system and we are able to consistently and reproducibly deliver a dose of 200-300 cfu. The reproducibility of the dose delivered to the lungs is shown for three separate experiments, in which the number of mycobacteria delivered to the lungs was quantitated 16 hours after infecting mice by the aerosol route with M. tuberculosis (Erdman) (FIG. 2, left panel). BALB/c mice were infected using the nose only aerosol delivery system and an inoculum of 200 cfu was delivered to the lungs. By day 21 post-infection, there was a 10,000 fold increase in the bacterial burden in the lung and dissemination to the spleen with a mycobacterial burden of nearly 10⁵ cfu (FIG. 2, middle panel). We have observed pronounced differences in the survival of susceptible and resistant inbred strains of mice following inoculation via the aerosol route, as have also been described by others (FIG. 2, right panel) (Medina, E. and North, R. J., Immunology, 93:270-274 (1998)).

We believe that aerosol inoculation is an important model for the study of the immune response to M. tuberculosis. It is a more physiological model (predictive of human disease) which has important implications for the study of immunity to tuberculosis. One of the critical differences between the intravenous and aerosol inoculations is that during intravenous inoculation, nearly a third of the inoculum is deposited in the spleen and triggers an immune response nearly immediately. In contrast, initial deposition of the inoculum in the airspace of the lung requires infected cells to migrate into the draining lymph nodes before initiation an adaptive immune response, which potentially allows the mycobacteria time for several replication cycles. This may explain why the respiratory route of infection is more lethal than the intravenous route (North, R. J., J Infect. Dis., 172:1550-1553 (1995); North, R. J. et al., Infect. Immun. 67:2010-2012 (1999)).

### Production of Cytokines by Pulmonary T Cells Correlates with Immunity to Tuberculosis

We propose that increased resistance to tuberculosis in α-galactosylceramide treated mice is due to the ability of α-galactosylceramide to alter the regulation of Th1/Th2 cell differentiation. Therefore, the production of cytokines by T cells from α-galactosylceramide treated and untreated mice using intracellular cytokine flow cytometry to characterize the mechanism of action of this immunomodulatory compound is performed in accordance with standard protocols and/or the methods provided herein.

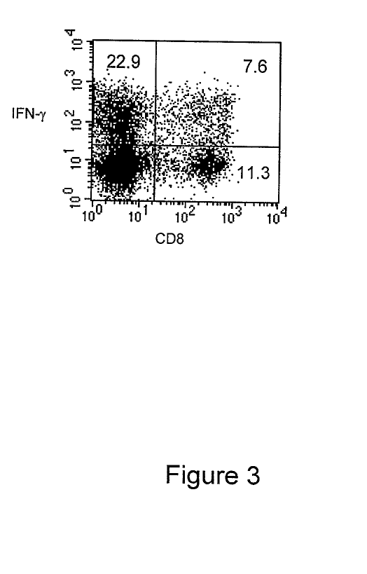

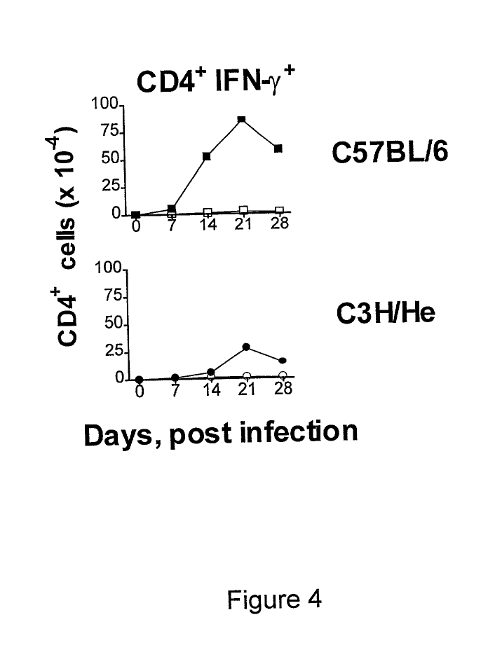

Intracellular flow cytometry permits the detection of cytokine production at the resolution of a single cell, and is particularly useful in the enumeration of cytokine producing cells. Furthermore, by coupling this technique with cell surface staining, the phenotype of the cytokine producing cells can be determined, even in a heterogeneous cell population (e.g., total splenocytes or lung mononuclear cells [MNCs]). By combining inhibitors of protein secretion (such as brefeldin A), which leads to intracellular accumulation of cytokines, and a brief stimulation of the cell populations ex vivo with PMA and ionomycin, which increases in the production of cytokines by previously committed cells, the technique has excellent sensitivity and specificity. To illustrate the utility of this technique, our studies using intracellular cytokine flow cytometry to compare the immune response of susceptible (C3H) and resistant (C57BL/6) murine strains after inoculation with M. tuberculosis are shown below (see FIGS. 3 and 4, and Chackerian, A. A., et al., Infect. Immun. 69(4):2666-74 (2001)).

Although the C3H and C57BL/6 splenic immune responses were quite similar and there was little change in the number or the percentage of cytokine producing cells during the first four weeks of infection, the pulmonary immune response to infection was significantly different. In C57BL/6 mice, there was an early and rapid influx of CD4⁺ T cells secreting IFN-γ into the lungs (FIG. 4). In C3H mice, the recruitment of IFN-γ producing CD4⁺ T cells into the lung was delayed and fewer cells were present at all time points analyzed. No IL-4 was detected during the first four weeks of infection, and although a small percentage of T cells produced IL-10, no differences between C57BL/6 and C3H/He mice were apparent. These results demonstrate that although the relative proportions of Th1 and Th2 cells are not significantly different (e.g., both murine strains mount immune responses dominated by Th1 cells), the early recruitment and increased number of cytokine producing cells in the lungs of C57BL/6 mice correlates with protective immunity. Such a difference was readily discernable using intracellular cytokine flow cytometry.

### The Absence of NKT Cells does not Impair Immunity to M. tuberculosis

While an important role for CD4⁺ T cells in immunity to tuberculosis has been clearly defined, the role of other T cell subsets, such as CD8⁺ T cells, is less clearly delineated. Some reports have shown that CD8⁺ T cells had a beneficial effect in immunity to tuberculosis, but others studies failed to show any role. We believe that the finding that β2 microglobulin (β2m) −/− mice, which lack MHC class I and consequently CD8⁺ T cells, had increased mortality following infection with M. tuberculosis suggests that CD8⁺ T cells play a critical role in the cellular immune response responsible for preventing development of tuberculosis (Flynn, J. L. et al., Proc. Natl. Acad. Sci. U.S.A., 89:12013-12017 (1992)).

As CD1 proteins also require β2m for their assembly and expression, we considered the possibility that the β2m dependence of the immune response to tuberculosis reflected a requirement for CD1 restricted T cells, rather than class I MHC restricted CD8⁺ T cells. Although mice lack homologues of human CD1a, -b, -c, and -e, they do have two CD1d genes, and hence are an excellent model for understanding the function of CD1d antigen presentation and role of CD1d restricted T cells. We therefore used TAP1−/− and CD1D −/− mice as models to test the relative importance of peptide and lipid antigen presentation pathways. These models had the potential to independently determine the significance of CD8⁺ T cells in immunity to M. tuberculosis.

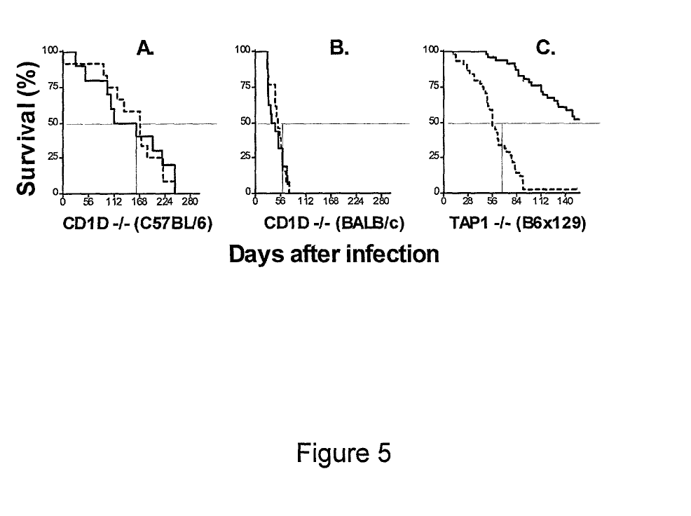

Our findings (Behar, S. M. et al, J. Exp. Med., 189:1973-1980 (1999)) demonstrated that CD1D −/− mice were no more susceptible to tuberculosis than control mice (FIG. 5AB). Since the absence of the CD1D1 and CD1D2 genes did not significantly alter the survival of mice, TAP1−/− mice were infected with M. tuberculosis to independently verify that the susceptibility of β2m −/− mice to tuberculosis was secondary to the absence of T cells restricted to MHC molecules loaded in the ER in a transporter dependent manner. The vast majority of such T cells are class I MHC restricted CD8⁺ T cells and mice with disruption of the TAP1 gene are known to have a profound deficiency in CD8⁺ T cells (Van Kaer, L. et al., Cell, 71:1205-1214 (1992)). Strikingly, the TAP1−/− mice were more vulnerable to death from infection compared to controls (p<0.0001 by the log-rank test) (FIG. 5C). This data supports our hypothesis of a critical role for TAP dependent antigen presentation for immunity to tuberculosis and a critical role for class I MHC restricted CD8⁺ T cells in the protective immune response to M. tuberculosis.

Although we failed to observe any difference in the susceptibility of CD1d −/− mice after intravenous inoculation with M. tuberculosis, we hypothesized that CD1d restricted NKT cells could still contribute to anti-mycobacterial immunity. This premise was based, in part, on our own observations that CD1d −/− mice had an increased mycobacterial burden in their lungs in some experiments. We considered the possibility that although NKT cells were not absolutely required for immunity to tuberculosis, their specific activation might enhance host defenses against M. tuberculosis. Therefore, we treated BALB/c mice with α-galactosylceramide, a reported known potent activator of NKT cells.

### Treatment with α-galactosylceramide Prolongs the Survival of Mice Inoculated with M. tuberculosis

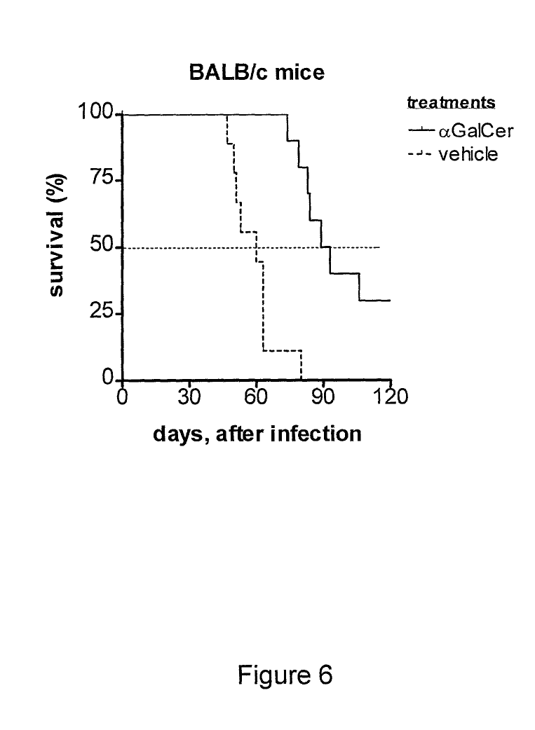

BALB/c mice were infected via the intravenous route with 5×10⁵ cfu of M. tuberculosis (Erdman). One day following the infection, the mice were randomly divided into two groups each containing nine mice, and treated with either α-galactosylceramide or the vehicle alone, using a protocol developed by Cui et al. (Cui, J. et al., Science 278(5343):1623-1626 (1997)). One day, five days, and nine days after infection, mice were administered 2 ug of α-galactosylceramide in 0.5 ml of PBS by intraperitoneal injection, or an equivalent amount of the vehicle in 0.5 ml of PBS as a control. Treatment of BALB/c mice with α-galactosylceramide resulted in increased survival compared to the control group (FIG. 6). While the mice treated with the vehicle alone had a mean survival time (MST) of 60 days, the treated group had a prolonged MST of 91 days (p<0.0001 by log-rank test). This experiment has been reproduced using BALB/c mice, using groups of nine mice, with similar results.

### D. Research Design and Methods

### (1). Evaluation of the Efficacy of α-galactosylceramide in the Treatment of Tuberculosis

### (a) α-galactosylceramide can Ameliorate Tuberculosis in Mice Inoculated with Virulent Mycobacterium tuberculosis by Either the Intravenous or Aerosol Routes of Infection

Susceptible mice are inoculated with virulent M. tuberculosis and then treated with either α-galactosylceramide or a control (vehicle alone) to evaluate the efficacy of this compound in protecting mice from disease. The following determinations are made: 1) whether α-galactosylceramide protects mice inoculated with M. tuberculosis by both the intravenous and the aerosol routes of infection; 2) whether α-galactosylceramide can be used to successfully treat mice with an established infection; and 3) the optimum dosing regimen for the administration of α-galactosylceramide.

Inbred mouse strains that are susceptible to tuberculosis are used in treatment trials to assess the role of α-galactosylceramide in the treatment of tuberculosis. Our preliminary data indicates that the administration of α-galactosylceramide prolongs the survival of mice that have been intravenously inoculated with M. tuberculosis (Erdman). In the experiments described herein, the BALB/c and C3HeB/FeJ murine strains are used to confirm and extend these findings. These mouse strains are susceptible to tuberculosis, and their inability to efficiently control mycobacterial infection results in a shortened life span compared to resistant murine strains (i.e., C57BL/6 mice). Furthermore, it is known, both from the literature and our own unpublished observations, that therapeutic interventions (i.e., IL-12 or traditional chemotherapy) can prolong the survival of these murine strains.

In each experiment, twenty mice are infected by either the intravenous or aerosol route. One day after infection, the mice are randomly divided into two groups. One group receives 100 ug/kg of α-galactosylceramide by IP injection on days 1, 5, and 9 after infection. The other group receives injections of the vehicle alone. The mice are weighed weekly and their health monitored. The survival of the α-galactosylceramide treated vs. untreated mice is analyzed. Details about the infection system, monitoring of the mice, and analysis of the data are outlined below.

An important question to address concerning the use of α-galactosylceramide in the treatment of tuberculosis is to assess its efficacy in treating established disease. Its beneficial effect, when administered starting one day after infection, compares favorably with other anti-mycobacterial therapeutic agents. Many studies evaluating the efficacy of new anti-mycobacterial antibiotics initiate treatment one day after infection, since the organisms have already invaded host cells, such as macrophages, within a few hours of inoculation (Miyazaki, E. et al., Antimicrob. Agents Chemother., 43:85-89 (1999)). However, a more rigorous evaluation would be to delay treatment until one or more weeks after infection (Kelly, B. P. et al., Antimicrob. Agents Chemother., 40:2809-2812 (1996); Klemens, S. P. and Cynamon, M. H., Antimicrob. Agents Chemother., 40:298-301, (1996)). Both approaches are evaluated using two different models of chronic tuberculosis.

In the first set of experiments, susceptible BALB/c mice are infected by the intravenous route with approximately 1-5×10⁵ cfu/mouse. This dose is lethal within 3-5 months following infection. To determine whether treatment can be delayed, two treatment arms are compared in each experiment: an immediate and a delayed treatment arm. Each arm consists of 10 mice treated with α-galactosylceramide compared to 10 mice treated with the vehicle alone. The immediate treatment arms receive vehicle or α-galactosylceramide on days 1, 5, and 9, after inoculation (see Preliminary Results). The delayed treatment arm commences treatment 3-4 weeks after infection. We have chosen this time point because inherently resistant strains generate a potent cell mediated immune response within one month, often resulting in a decline or a plateau in the tissue mycobacterial burden. In genetically susceptible mouse strains, the mycobacterial burden in the lungs continues to gradually increase, and the animals' health will slowly decline, with ultimate death from overwhelming disease. Even in genetically resistant murine strains, the mycobacterial burden in the lungs will gradually increase during the several months following infection, a phase of disease akin to recrudescence (Orme, I. M., J. Immunol., 138:293-298 (1987)). We propose that administration of α-galactosylceramide at this point during the natural history of the disease will reactivate the immune response leading to improved control of the infection. As, endpoints, body weight, as a gross measure of the health of the animals, and survival, are monitored. In some experiments, inherently resistant C57BL/6 mice also are treated starting 4 weeks after infection, during the commencement of the plateau/recrudescence phase of the infection. In this case, because of the prolonged survival of these mice, bacterial burden in the various target organs (lungs, spleen, and liver) are monitored at several time points after treatment, instead of survival.

Lastly, we note that α-galactosylceramide prolongs the survival of mice but does not appear (based on preliminary results) to cure them of tuberculosis. This may be due to the short lived effect of α-galactosylceramide compared to the persistent nature of infection with M. tuberculosis. The dosing regimen that we have used was modeled after one shown to induce anti-tumor immunity. Those studies were of short duration, especially compared to the experiments proposed herein (Cui, J. et al., Science 278(5343):1623-1626 (1997)). Therefore, the effect of altering the dosing regimen is examined. Our first modification is to administer α-galactosylceramide for a longer duration. Instead of stopping on day 9, its administration is continued on every fourth day for the first month. Depending on the results, other dosing regimens also are tried (i.e., weekly dosing regimens, etc.).

Bacteria. Virulent M. tuberculosis (Erdman strain; originally obtained from Barry Bloom, Albert Einstein College of Medicine, Bronx, N.Y.) was passed through C57BL/6 mice, grown in Middlebrook 7H9 supplemented with oleic acid-albumin-dextrose complex (OADC) (Difco), before freezing at −80° C. Before inoculation of mice, an aliquot is thawed, sonicated twice for 10 seconds using a cup horn sonicator, triturated using a 30G needle, and then diluted in normal saline (0.9% NaCl) containing 0.02% Tween-80. In addition to titering each batch of bacteria, the actual inoculating dose is determined for each experiment by plating serial dilutions from the aliquot of the thawed bacteria onto 7H10 agar plates and enumerating the colony forming units (cfu) three weeks later.

Intravenous inoculation method. Mice are infected intravenously via the lateral tail vein using an inoculum of between 10⁵ and 10⁶ live mycobacteria.

Aerosol inoculation method. Inoculation of mice by the inhalation route is done using an INTOX nose only exposure unit (Intox Products, Albuquerque N.Mex.). A suspension of M. tuberculosis is made in 10 ml saline plus 0.02% Tween-80 and loaded into the nebulizer (MiniHEART nebulizer, VORTRAN Medical Technologies). Animals then are loaded into the exposure chamber. The system is run for 20 minutes during which time the mice are exposed to the bacterial aerosol. Then, the nebulizer air flow is shut off and the system allowed to purge with fresh air for 5 minutes before removing the animals. Total exposure time is approximately 25 minutes. Appropriate preliminary experiments have been done to establish the concentration of M. tuberculosis needed in order to deliver a dose of 200-300 cfu, an inoculum we have chosen based on its ability to cause progressive lung pathology (Saunders, B. M. et al., Infect. Immun., 66:5508-5514 (1998)). The total number of bacteria actually deposited in the lungs is determined from colony counts of whole lung homogenates within 16 hours after inoculation for five mice from each experimental group. The entire lung of each mouse is homogenized into 1 cc of normal saline/Tween80 and 0.2 cc are plated on 7H10 agar plates. When an additional 10-fold dilution is plated, this method allows the accurate enumeration of the loading inoculum of M. tuberculosis in the range of between 5-5000 cfu/lung.

Mice. Mice are obtained from Jackson Laboratories (Bar Harbor, Me.) or from colonies maintained at the Animal Resource Division of the Dana Farber Cancer Institute. Mice 6-10 weeks of age of both genders are used in the proposed experiments. In any given experiment, only mice of the same age (within one week) and of the same gender are used. All infected mice are housed under specific pathogen free conditions in the Animal Biohazard Containment Suite (a biosafety level 3 facility at Dana Farber Cancer Institute, Boston, Mass.) and used in a protocol approved by the institution.

Administration of α-galactosylceramide. In our preliminary experiments, we adapted the protocol for the administration of α-galactosylceramide from the work of Taniguchi et al. (Cui, J. et al., Science 278(5343):1623-1626 (1997)). Based on their tumor model, we administered 100 ug/kg α-galactosylceramide (Kirin Pharmaceutical Research Laboratory, Gunma, Japan), intraperitoneally on days 1, 5, and 9 after infection. For a typical 20 gm mouse, the dose is therefore 2 ugs. The vehicle is 0.5% polysorbate 20 and when properly diluted (in PBS), the mice receive 0.5 ml of 0.01% polysorbate 20. While this schedule resulted in a pronounced biological effect, we plan to examine this important variable in the treatment of mice infected with M. tuberculosis (see below).

Survival studies. Mice inoculated with M. tuberculosis are monitored for survival. In general, 10 mice per experimental group are used (range, 8-15). Infected mice are checked daily for signs of morbidity including, but not limited to, failure to eat, drink, or right themselves in response to lateral recumbency, and general appearance. The presence of one or more of these signs is an indication for euthanasia of the animal by CO₂ inhalation.

Data analysis. Each group includes 10 mice, matched for gender and age. The results are analyzed using the method of Kaplan and Meier, and the curves for each treatment group are compared using the log-rank test. The statistics are calculated using the Prism software package (GraphPad, San Diego, Calif.). Additional statistical consultation is obtained as needed from the Brigham and Women's Hospital Biostatistics Consulting Service or from the Multipurpose Arthritis & Musculoskeletal Diseases Center.

### (b) Determine the Efficacy of Treatment with α-galactosylceramide with Respect to a Reduction in the Numbers of Bacteria Found in Infected Target Organs

To better understand why treatment with α-galactosylceramide leads to the prolonged survival of M. tuberculosis infected treated mice, the control of the mycobacterial infection in the lung, spleen, and liver is assessed by determining the mycobacterial burden in these organs during the course of infection. Although the experience of many other investigators, including ourselves, is that lower colony counts parallel increased survival after M. tuberculosis infection (Behar, S. M. et al, J. Exp. Med., 189:1973-1980 (1999)), enhanced survival may reflect less severe tissue pathology (i.e., decreased hypersensitivity reaction), rather than better control of the mycobacterial infection. The kinetics of the resolution of the infection may also provide clues concerning the role of NKT cells in infection. For example, the TCRβ, IFN-γ, IL-1, IL-6, IL-12, β2m, and TAP1 knockout mice all have increased mortality and increased mycobacterial burden compared to appropriate control mice (Flynn, J. L. et al., Proc. Natl. Acad. Sci. U.S.A., 89:12013-12017 (1992); Ladel, C. H. et al., [published erratum appears in Eur. J. Immunol., (12):3525 (Dec. 25, 1995)], Eur. J Immunol., 25:2877-2881 (1995); Cooper, A. M. et al., J. Exp. Med., 186:39-45 (1997); Flynn, J. L. et al., Immunity, 2:561-572 (1995)). In contrast, the TCRδ−/− mouse has increased mortality, despite having similar mycobacterial burden in the lungs, spleen, and liver compared to TCRδ+/+ mice (D'Souza et al., J. Immunol., 158:1217-1221 (1997)). The increased severity of pulmonary pathology seen in the TCRδ−/− mouse, despite similar mycobacterial burden, suggests that γδ T cells have an anti-inflammatory role (D'Souza et al., J. Immunol., 158:1217-1221 (1997)). Colony count data from untreated and α-galactosylceramide treated mice provide insight concerning how α-galactosylceramide modifies the immune response.

Colony-forming unit determination. Mice are infected by the intravenous or aerosol route so that ˜200-300 cfu are deposited in the lungs. The actual number of bacteria deposited in the lungs of the infected mice is determined by sacrificing several mice on the day following aerosol inoculation and enumerating the number of cfu in the lungs. Then, groups of 5-8 mice per condition are sacrificed 1, 2, 3, 4, and 6 weeks after infection. Depending on the survival of the mice, later time points may be examined in some experiments. The left lung, middle liver lobe, and ½ spleen are homogenized in 0.9% NaCl/0.02% Tween80 using Teflon homogenizers. The remaining portions of the organs are examined histologically after fixation or used to prepare MNCs that will be used to analyze T cell cytokine production. The bacterial burden in the lung, spleen, and liver are determined by plating 10-fold serial dilutions of tissue homogenates on 7H10 agar plates and counting colonies after a three week incubation at 37° C.

Data analysis. At each time point, 5-8 mice per condition are analyzed. The colony counts from the untreated and α-galactosylceramide treated mice are compared to each other using a nonparametric test that compares two unpaired groups (the Mann-Whitney test). A two-tail P value is calculated using the Prism software package (GraphPad, San Diego, Calif.).

### (c) Analysis of the Pathology of Tuberculosis Following Treatment with α-galactosylceramide

Microbial pathogens can cause tissue pathology by their direct toxic effect on cells, or as a consequence of the inflammatory reaction and the immune response that they elicit (i.e., via a hypersensitivity reaction). For example, some of the worse complications of Pneumocystis carnii infection are the consequences of an intense pulmonary inflammatory reaction that is by the elicited organism. Corticosteroids paradoxically have been found to be beneficial in the treatment of disease by reducing the host inflammatory response. Similarly, the pathology of tuberculosis is in large part due to the persistent and chronic nature of the host response, as opposed to the capacity of M. tuberculosis to directly damage host cells.

In human tuberculosis patients and guinea pigs experimentally infected with M. tuberculosis, granulomas are composed of aggregates of epithelioid cells (i.e., activated macrophages) surrounded by concentric layers of lymphocytes and fibroblasts. In advance disease, the granuloma centers undergo caseous necrosis, and ultimately, in individuals that develop immunity, they undergo extensive fibrosis and calcification. Mice, generally thought to be a resistant species, have slightly different pathology which may be a reflection of their immunity. Orme and his colleagues have reported that the inflammatory response in the lung is characterized by cords of infiltrating lymphocytes that penetrate into the center of the lesion, instead of remaining in the outer rim (Orme, I. M., Trends. Microbiol. , 6:94-97 (1998)). As described above, NKT cells may be important in modulating pulmonary inflammation since in their absence, granulomas reportedly fail to develop under certain conditions (Apostolou, I. et al., [published erratum appears in Proc. Natl. Acad. Sci. U S.A., 96(13):7610], Proc. Natl. Acad. Sci. U.S.A., 96:5141-5146 (Jun. 22, 1999)). Although we observed normal granuloma formation in CD1d −/− mice infected with M. tuberculosis, it remains a possibility that mice treated with α-galactosylceramide may have more efficient granuloma formation.

The pathology of the lung and liver from infected α-galactosylceramide treated and untreated mice are examined to characterize the inflammatory response and to understand the basis, or the consequences, of the increased resistance of α-galactosylceramide treated mice to tuberculosis. The lung has been chosen for examination since it is the principal site of disease; the liver also is examined because there it is easy to identify well-formed granulomas. Portions of the lungs and liver are stained with hematoxylin and eosin and examined histologically. In addition, samples from these tissues are stained for acid-fast bacilli (AFB). This independent measure of mycobacterial burden is used to confirm the results obtained by the determination of colony counts from organ homogenates. We have carried out similar studies (Behar, S. M. et al., J. Exp. Med., 189:1973-1980 (1999), and Chackerian et al., Infect. Immun. 69(4):2666-74 (2001)) which have been facilitated by the Histopathology Core in the Animal Resource Division at the Dana Farber Cancer Institute (Boston, Mass.).

Pathology. At each time point, samples of tissue obtained for histological analysis are immediately fixed in 10% buffered formalin and then embedded in paraffin or JB-4 plastic resin blocks. Sections (2 um) are stained with hematoxylin and eosin for routine histopathological analysis or by the Fite-Faraco method for AFB (Fite, G. L. et al., Arch. Pathol., 43:624-625 (1947)).

### (d) Assess the Effect of Treatment with α-galactosylceramide on T Cell Cytokine Production

We believe that the increased resistance of α-galactosylceramide treated mice to tuberculosis is likely to result from an alteration in the immunoregulation of the Th1/Th2 balance. This aim characterizes the changes in the immune response to M. tuberculosis in α-galactosylceramide treated and untreated mice to provide insight into how α-galactosylceramide modifies the susceptibility of mice to tuberculosis.

To detect a change in the Th1/Th2 balance in the lung (and the spleen) during the immune response to tuberculosis, this laboratory has been using intracellular cytokine flow cytometry which can determine both the phenotype of the T cells present, as well as their function as measured by cytokine production. This technique is sensitive, quantitative, and can be safely carried out in the BL-3 setting. In our experiments using C3H/He (susceptible) and C57BL/6 (resistant) mice, the susceptibility of the C3H mice correlated with a failure to recruit cytokine producing CD4⁺ T cells to the lungs, instead of a change in the balance of Th1/Th2 cytokine producing T cells.

Groups of α-galactosylceramide treated and untreated mice are sacrificed at various times after infection with M. tuberculosis and mononuclear cells are isolated from the lungs and spleens. The absolute number and percentage of splenic and pulmonary CD4⁺ and CD8⁺ T cells that are producing Th1 cytokines (IFN-γ), Th2 cytokines (IL-4, -5, and -10), as well as TNF-α and GM-CSF, are quantitated.

Intracellular cytokine cytometric analysis. The production of cytokines by T cells isolated from the lung and spleen is determined by intracellular cytokine flow cytometry. MNCs are isolated and pooled from the spleens and lungs of infected mice using our established techniques (see above and Preliminary results). Cells are cultured in media with brefeldin A 10 ug/ml for 3.5 hours in the presence (activated condition) or absence (unstimulated condition) of PMA (10 ng/ml) and ionomycin (1 ug/ml). The brefeldin A leads to the accumulation of intracellular cytokines by blocking intracellular transport. After culture, the cells are washed, the Fc receptors are blocked using the 2.4G2 mAb, and then staining is done with either directly conjugated (Alexa488) or biotinylated monoclonal antibodies to T cell markers (including CD4, CD8, and CD45RB) and other cell surface proteins. When biotinylated mAbs are used, a suitable secondary reagent is used such as Cy5-streptavidin. After extensive washing, the cells are fixed overnight with 1% paraformaldehyde to kill any viable bacteria. The following day, cells are permeabilized (Pemeabilization Media B; Caltag) and stained with phycoerythrin conjugated anti-cytokine antibodies in for 20 minutes at room temperature. The cells are analyzed using a FACSort (Becton Dickinson). Absolute cell numbers are derived from the cell counts and cytometric analysis. The final data is normalized to a “per mouse” basis.

### (2). Assess the Efficacy of α-galactosylceramide Acting Synergistically With Traditional Anti-tuberculous Chemotherapy

The immunomodulatory effect of α-galactosylceramide appears to enhance protection from tuberculosis; however, ultimately, all of the infected mice died despite treatment. This indicates that, under the conditions used on our preliminary experiments, α-galactosylceramide did not result in a bacteriological cure. Therefore, we envision that α-galactosylceramide will have a role in the adjunctive treatment of tuberculosis. It may be particularly useful in patients with MDR tuberculosis or in patients with compromised immune systems, such as patients with AIDS. Therefore, we propose the following experiment to assess the synergistic effect of α-galactosylceramide with conventional anti-tuberculous chemotherapy.

Female BALB/c mice (5-6 weeks of age) are infected by the intravenous route using 10⁶ cfu/mouse. On the day following infection, the mice are randomly divided into four groups of 12 mice each. Each group receives treatment starting on day seven following infection. Every mouse receives isoniazid (INH) or water by gavage (five days a week). In addition, every mouse receives α-galactosylceramide or vehicle by intraperitoneal injection on days 7, 11 and 15 after infection. The following groups are studied:

| | | | By intraperitoneal |
|---|---|---|---|
| | | | injection on d7, |
| | (n) | By daily gavage | 11, & 15 |
| 1 | 12 | Control, Water | Vehicle |
| 2 | 12 | Control, Water | α-galactosylceramide |
| 3 | 12 | Chemotherapy, INH (25 mg/kg/day) | Vehicle |
| 4 | 12 | Chemotherapy, INH (25 mg/kg/day) | α-galactosylceramide |

Treatment is continued for up to six weeks. Three and six weeks following infection groups of eight mice are sacrificed. The mice are weighed and the lung and spleen colony counts determined as described above. The design of this experiment is similar to ones used to test new antibiotics for anti-mycobacterial activity (Miyazaki, E. et al., Antimicrob. Agents Chemother., 43:85-89 (1999); Klemens, S.P. and Cynamon, M.H., Antimicrob. Agents Chemother., 40:298-301, (1996)).

### Example 2: Assays for the Analysis of Immune Alteration

One measure of immune alteration is modulation of T cell products. To measure T cell products including cytokines, purified T cells from a variety of sources including peripheral blood or solid organs, or in vitro grown T cell lines, clones, or T-T hybridomas, are be cultured with the putative agent (i.e., α-glycosylceramides or libraries of potential agents), in the presence of an antigen presenting cell (e.g., CD1d transfected tumor cell lines or native antigen presenting cells such as macrophages or dendritic cells that express CD1d). Instead of antigen presenting cells, purified CD1d protein can also be used (Gumperz, J. E. et al., Immunity, 12(2):211-221 (February 2000)). Supernatant from these cultures is sampled 24-72 hours later and the amount of secreted cytokine found therein is quantitated by ELISA using standard techniques and commercially available reagents. (Gumperz, J. E. et al., Immunity, 12(2):211-221 (February 2000); (Behar, S. M. et al., J. Immunol., 162:161-167 (1999); Behar, S. M. et al., J. Exp. Med., 182:2007-2018 (1995); Behar, S. M. et al., Arthritis Rheum. 41:498-506 (1998)).

To determine whether other components of the immune system are activated as a consequence of α-glycosylceramide treatment, one determines whether cell surface markers are upregulated specifically after treatment with the putative agent (but not in the absence of treatment, nor after treatment with a control agent). This is done by flow cytometry using cells from the treated individual (animal or human). Standard techniques exist to analyze different immune cell subsets (B cells, NK cells, T cells, macrophages, dendritic cells, neutrophils, etc.). For example, flow cytometry can be used to determine whether these cell types express cell surface proteins such as MHC molecules, cytokine receptors, activation markers (i.e., CD69), or whether these cells produce cytokines (e.g., intracellular cytokine production as determined by intracellular cytokine flow cytometry), as an indication of their activated phenotype. (Camaud, C. et al., J. Immunol., 163:4647-4650 (1999)).

### SUMMARY

The ability of α-galactosylceramide to prolong the survival of mice after intravenous inoculation with tuberculosis is a remarkable and unexpected finding. Furthermore, although applicants do not intend to be bound to any particular theory or mechanism, since its proposed mechanism of action is the activation of immunoregulatory CD1d restricted NKT cells, we believe that such a compound may have a beneficial synergistic effect when combined with traditional anti-mycobacterial chemotherapy. The murine model is an excellent system to investigate the effect of α-galactosylceramide on M. tuberculosis infection since both CD1d and NKT cells are conserved structurally and functionally between mice and humans, i.e., the animal model that we have used is predictive of an in vivo human effect. We believe that the experiments disclosed herein can be used to evaluate the use of α-galactosylceramide in the treatment of tuberculosis, based on our expertise both in the field of CD1 and in murine models of tuberculosis. Following the demonstration of a role for α-galactosylceramide in the treatment of murine tuberculosis, translation into clinical trials should be facilitated by the fact that α-galactosylceramide is relatively nontoxic and is currently in human trials for the cancer immunotherapy.

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

Documents that are specifically incorporated in their entirety herein by reference include:

EP 0957161A1, entitled “Method for Activating Human Antigen Presenting Cells, Activated Human Antigen Presenting Cells, and Use of the Same,” Publication no. WO 98/29534, published Jul. 9, 1998 (referred to herein as “Kirin European Application”); Kirin U.S. Pat. No. 5,936,076, entitled “alphaGalactosyl Derivatives”, issued Aug. 10, 1999 (referred to herein as “Kirin U.S. Pat. No. 5,946,076”); and Chackerian et al., Infect. Immun. 69(4):2666-74 (2001).

### Table 1. Introduction

Table 1 contains various compounds that are useful for practicing the methods of the invention. In general, these compounds are based on the structure having formula (A):

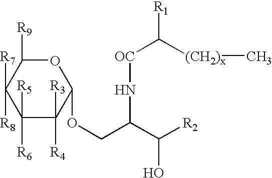

wherein R₁ to R₉ and X are to be defined later. Exemplary glycoside compounds include: (2S, 3S, 4R)-1, -(α-D-galactopyranosyloxy)-2-hexacosanoylamino-3,4-octadecanediol or a salt thereof.

More specifically, the compounds of formula (A) include:

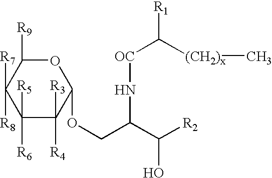

wherein:

R₁ is H or OH;

X is an integer of from 7 to 25;

R₂ is a substituent defined by any one of the following (a) to (e):

(a)—CH₂(CH₂)_{y}CH₃;

(b)—CH(OH)(CH₂)_{y}CH₃;

(c)—CH(OH)(CH₂)_{y}CH(CH₃)₂;

(d)—CH═CH(CH₂)_{y}CH₃; and

(e)—CH(OH)(CH₂)_{y}CH(CH₃)CH₂CH₃;

wherein Y is an integer of from 5 to 17;

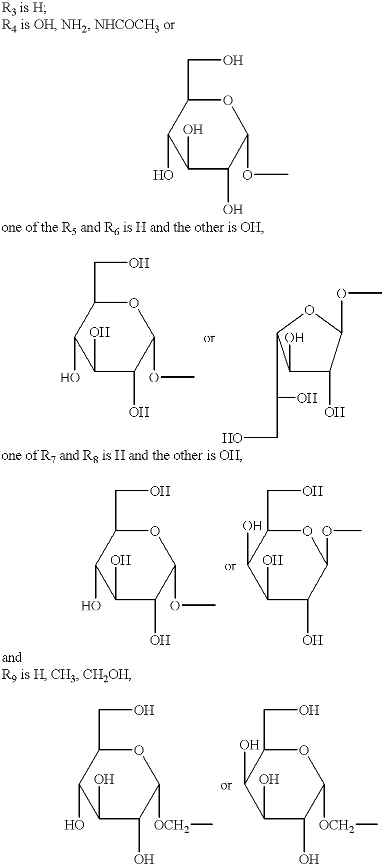

The glycoside compounds also embrace the compound represented by Formula (B) or salts thereof:

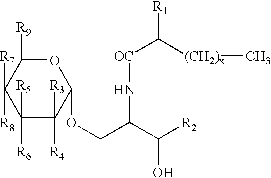

wherein:

R₁, X and R₂ are as defined as in the case of Formula (A); and

R₃ to R₉ are substituents defined by any one of the following (i) to (iii):

(i) [galactose type]

each of R₃, R₆ and R₈ is H;

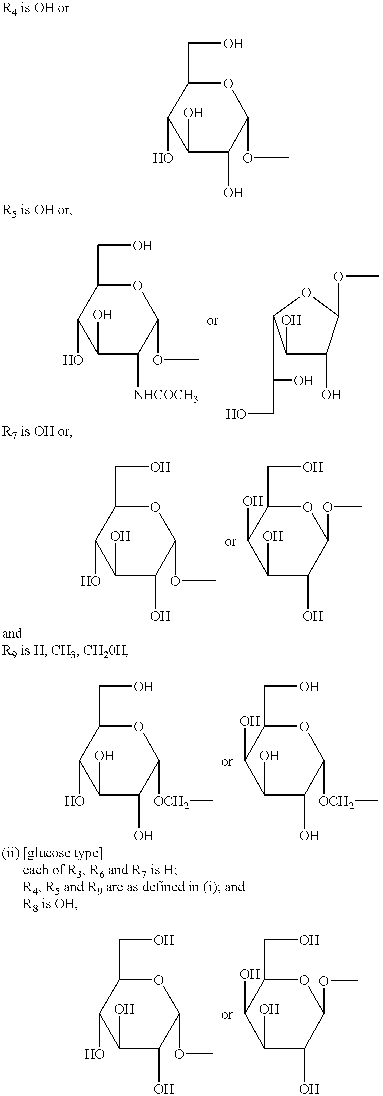

or

(iii) [allose type]

each of R₃, R₅ and R₇ is H;

each of R₄, R₆ and R₈ is OH; and

R₉ is H, CH₃ or CH₂OH.

The glycoside compounds defined by formula (A) or (B) above are comprised of a sugar moiety and an aglycone moiety, and some of them are also referred to as α-cerebrosides, α-glycosylceramides, α-glucosylceramides, α-galactocerebrosides or α-galactosylceramides. These compounds are characterized by having the α-form of anomeric configuration.

In the glycoside compound, the sugar moiety is preferably of [galactose type] as defined in (i), and more preferably of one wherein each of R₃, R₆ and R₈ is H, each of R₄, R₅ and R₇ is OH and R₉ is CH20H (i.e., α-galactopyranosyl).

In the glycoside compound, the aglycone moiety preferably has R, being any one of the substituents (b), (c) and (e) above, and more preferably has R₁ being H (i.e., kerasin type) and R₂ being the substituent (b). X is preferably an integer of 21 to 25 and Y is preferably an integer of 11 to 15. 5 Preferable examples of the glycoside compound of the present invention are listed below. In the list, compounds (1)-(9), (10)-(24), (25)-(31), (32)-(33), and (34) are those compounds in which R₂ is the substituent (a), (b), (c), (d) or (e) above, respectively. The alphabet letters A, B, C and D behind the compounds' name indicate the reference specifications of WO93/05055, WO94/02168, WO94/09020 and WO94/24142, respectively, which describe the synthesis methods of the annoted compounds. Among the glycoside compounds below, compound (14) (2S,3S,4R)-1-(α-D-galactopyranosyloxy)-2-hexacosanoylamino-3,4-octadecanediol (referred to as “KRN7000” hereinbelow), is most preferable. With respect to this compound, an example of the synthesis process is illustrated in the Production Example and Scheme 1 of EPO 957161A1.

TABLE 1

| | |
|---|---|
| (1) (2S, 3R)-1-(α-D-galactopyranosyloxy)-2-[(R)-2- | A |
| hydroxtetracosanoylamino]-3-octadecanol | |
| (2) (2S, 3R)-1-(α-D-galactopyranosyloxy)-2-tetracosanoyl- | A |
| amino]-3-octadecanol | |
| (3) (2S,3R)-1-(α-D-galactopyranosyloxy)-2-tetradecanoyl- | A |
| amino-3-octadecanol | |
| (4) (2S,3R)-1-(α-D-glucopyranosyloxy)-2-tetradecanoyl- | C |
| amino-3-octadecanol | |
| (5) (2S,3R)-1-(6′-deoxy-α-D-galactopyranosyloxy)-2- | C |
| tetradecanoylamino-3-octadecanol | |
| (6) (2S,3R)-1-(B-L-arabinopyranosyloxy)-2-tetradecanoyl- | C |
| amino-3-octadecanol | |
| (7) (2S,3R)-1-(α-D-galactopyranosyloxy)-2-tetradecanoyl- | A |
| amino-3-hexadecanol | |
| (8) (2R,3R)-1-(α-D-galactopyranosyloxy)-2-tetradecanoyl- | A |
| amino-3-hexadecanol | |
| (9) (2R,3S)-1-(α-D-galactopyranosyloxy)-2-tetradecanoyl- | A |
| amino-3-hexadecanol | |
| (10) (2S,3S,4R)-1-(α-D-galactopyranosyloxy)-2-[(R)-2- | A |
| hydroxtetracosanoylamino]-3,4-octadecanediol | |
| (11) (2S,3S,4R)-1-(α-D-galactopyranosyloxy)-2-[(R)-2- | A |
| hydroxtetracosanoylamino]-3,4-undecanediol | |
| (12) (2S,3S,4R)-1-(α-D-galactopyranosyloxy)-2-[(R)-2- | A |
| hydroxyhexacosanoylamino]-3,4-icosanediol | |
| (13) (2S,3S,4R)-1-(α-D-galactopyranosyloxy)-2-[(S)-2- | A |
| hydroxtetracosanoylamino]-3,4-hep-tadecanediol | |
| (14) (2S,3S,4R)-1-(α-D-galactopyranosyloxy)-2- | EPO |
| hexacosanoylamino-3,4-o˜adecanediol | 957161 A1 |
| | Production |
| | Ex. |
| (15) (2S,3S,4R)-1-(α-D-galactopyranosyloxy)-2- | B |
| octacosanoylamino-3,4-heptadecanediol | |
| (16) (2S,3S,4R)-1-(α-D-galactopyranosyloxy)-2- | A |
| tetracosanoylamino-3,4-octadecanediol | |
| (17) (2S,3S,4R)-1-(α-D-galactopyranosyloxy)-2- | A |
| tetracosanoylamino-3,4-undecanediol | |
| (18) (2S,3S,4R)-1-(α-D-galactopyranosyloxy)-2- | C |
| hexacosanoylamino-3,4-o-adecanediol | |
| (19) 0-β-D-galactofuranosyl-(1→3)-O-α-D- | D |
| galactopyranosyl-(1→1I)-(2S,3S,4R)-2-amino-N- | |
| [(R)-2-hydroxytetracosanoyl]-1,3,4-octadecanetriol | |
| (20) O-α-D-galactopyranosyl-(1→6)-O- | D |
| α-D-glucopyranosyl-(1→1)-(2S,3S,4R)-2-amino-N- | |
| hexacosanoyl-1,3,4-octadecanetriol | |
| (21) O-α-D-galactopyranosyl-(1→6)-O-α-D- | D |
| galactopyranosyl-(1→1)-(2S,3S,4R)-2-amino-N- | |
| hexacosanoyl-1,3,4-octadecanetriol | |
| (22) O-α-D-glucopyranosyl-(1→4)-O-α-D- | D |
| glucopyranosyl-(1→1)-(2S,3S,4R)-2-amino-N- | |
| hexacosanoyl-1,3,4-octadecanetriol | |
| (23) O-(N-acetyl-2-amino-2-deoxy-α-D-galactopyranosyl- | D |
| (1→3)-O-[α-D-glucopyranosyl-(1→2)]-O-α-D- | |
| galactopyranosyl-(1→1)-(2S,3S,4R)-2-amino-N- | |
| [(R)-2-hydroxyhexacosanoyl-1,3,4-octadecanetriol | |
| (24) O-(N-acetyl-2-amino-2-deoxy-α-D- | D |
| galactopyranosyl-(1→3)-O-[α-D-glucopyranosyl- | |
| (1→2)]-O-α-D-galactopyranosyl-(1→1)-(2S,3S,4R)-2- | |
| amino-N-[(R)-2-hydroxytetracosanoyl-1,3,4-hexadecanediol | |
| (25) (2S,3S,4R)-1-(α-D-galactopyranosyloxy)-2- | A |
| [(R)-2-hydroxtricosanoylamino]-16-methyl-3,4- | |
| heptadecanediol | |
| (26) (2S,3S,4R)-1-(α-D-galactopyranosyloxy)- | A |
| 2-[(S)-2-hydroxtetracosanoylamino]-16- | |
| methyl-3,4-heptadecanediol | |
| (27) (2S,3S,4R)-1-(α-D-galactopyranosyloxy)-I | A |
| 6-methyl-2-tetracosanoylaminol-3,4-hepta-decanediol | |
| (28) O-β-D-galactofuranosyl-(I +3)-O-α-D- | D |
| galactopyranosyl-(1 +I)-(2S,3S,4R)-2-amino-N- | |
| [(R)-2-hydroxytetracosanoyll-I 7-methyl-1,3,4- | |
| octadecanetriol | |
| (29) O-β-D-galactofuranosyl-(I +3)-O-α-D- | D |
| galactopyranosyl-(1 +I)-(2S,3S,4R)-2-amino-N- | |
| [(R)-2-hydroxytetracosanoyl]-15-methyl-1,3,4- | |
| hexadecanediol | |
| (30) O-(N-acetyl-2-amino-2-deoxy-α-D-galactopyranosyl- | D |
| (1 ÷3)-O-[α-D-glucopyranosyl-(1→2)]-O-α-D- | |
| galactopyranosyl-(1→1)-(2S,3S,4R)-2-amino-N- | |
| [(R)-2-hydroxyhexacosanoyl-16-methyl-1,3,4- | |
| octadecanetriol | |
| (31) O-(N-acetyl-2-amino-2-deoxy-α-D-galactopyranosyl- | D |
| (I +3)-O-[α-D-glucopyranosyl-(1→2)]-O-α-D- | |
| galactopyranosyl-(1→1)-(2S,3S,4R)-2-amino-N-[(R)-2- | |
| hydroxytetracosanoyl-16-methyl-1,3,4- | |
| heptadecanetriol | |
| (32) (2S,3S,4E)-1-(α-D-galactopyranosyloxy)-2- | A |
| octadecanoylamino-4-octadecene-3-ol | |
| (33) (2S,3S,4E)-1-(α-D-galactopyranosyloxy)-2- | A |
| tetradecanoylamino-4-octadecene-3-ol | |
| (34) (2S,3S,4R)-1-(α-D-galactopyranosyloxy)-2-[(R)- | A |
| 2-hydroxypentacosanoylamino]-16-methyl-3,4- | |
| octadecanediol | |

The glycoside compound defined by formula (A) or (B) may form an acid addition salt with a pharmaceutically acceptable acid. Examples of the acid to be used for formation of such an acid addition salt include inorganic acids such as hydrochloric acid, sulfuric acid, nitric acid and phosphoric acid; and organic acids such as acetic acid, propionic acid, maleic acid, oleic acid, palmitic acid, citric acid, succinic acid, tartaric acid, fumaric acid, glutamic acid, pantothenic acid, lauryl sulfonic acid, methanesulfonic acid and phthalic acid.

### Table 2. Introduction

The α-galactosylceramides according to the present invention are represented by the following formula (A):

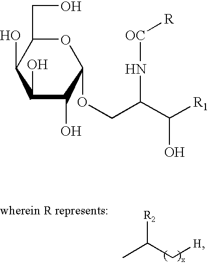

Where R₂ represents H or OH and X denotes an integer of 0-26, or R represents —(CH₂)₇ CH═CH(CH₂)₇CH₃ and R₁ represents any one of the substituents defined by the following (a)-(e):

(a)—CH₂(CH₂)_{Y}CH₃,

(b)—CH(OH)(CH₂)_{Y}CH₃,

(c)—CH(OH)(CH₂)_{Y}CH(CH₃)₂,

(d)—CH═CH(CH₂)_{Y}CH₃, and

(e)—CH(OH)(CH₂)_{Y}CH(CH₃)CH₂CH₃,

where Y denotes an integer of 5-17.

In the aforementioned formula (A),

(1) the compound in which R represents

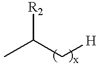

is represented by the formula (1):

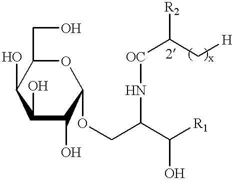

and (2) the compound in which R represents —(CH₂)₇CH═CH(CH₂)₇CH₃ is represented by the formula (XXI):

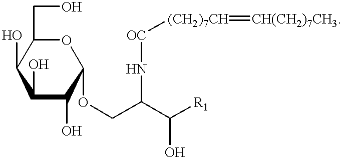

Exemplary α-galactosylceramides represented by the formula (I) are specified below:

(1) (2S,3 S,4R)-1-α-D-galactopyranosyloxy)-2-[(R)-2-hydroxytetracosanoylamino]-3,4-heptadecanediol,

(2) (2S,3S,4R)-1-(α-D-galactopyranosyloxy)-2-[(R)-2-hydroxytetracosanoylamino]-3,4-hexadecanediol,

(3) (2S,3 S,4R)-1-α-D-galactopyranosyloxy)-2-[(R)-2-hydroxytricosanoylamino]-16-methyl-3,4-heptadecanediol, and

(4) (2S,3S,4R)-1-α-D-galactopyranosyloxy)-2-[(R)-2-hydroxytricosanoylamino]-16-methyl-3,4-octadecanediol.

The compound of the present invention represented by the formula (A) (i.e. formula (I) and (XXI) can be also synthesized chemically according to the reaction route schemes described in U.S. Pat. No. 5,936,076.

The α-galactosylceramides according to the present invention, as described above, are represented by the formula (A) (i.e. formula (I) and (XXI), and R₁ in the formula (I) is preferably represented by the following (a)-(e):

(a) —CH₂(CH₂)_{Y}CH₃,

wherein, when R₂ represents H, it is preferable that X denote an integer of 0-24 and Y denote an integer of 7-15; when R₂ represents OH, it is preferable that X denote an integer of 20-24 and Y denote an integer of 11-15; when R₂ represents H, it is particularly that X denote an integer of 8-22 and Y denote an integer of 9-13; and when R₂represents OH, it is particularly preferable that X denote an integer of 21-23 and Y denote an integer of 12-14;

(b) —CH(OH)(CH₂)_{Y}CH₃,

wherein, when R₂ represents H, it is preferable that X denote an integer of 18-26 and Y denote an integer of 5-15; when R₂ represents OH, it is preferable that X denote an integer of 18-26 and Y denote an integer of 5-17; further when R₂ represents H, it is particularly preferable that X denote an integer of 21-25 and Y denote an integer of 6-14; and when R₂ represents OH, it is particularly preferable that X denote an integer of 21-25 and Y denote an integer of 6-16;

(c) —CH(OH)(CH₂)_{Y}CH(CH₃)₂,

wherein, when R₂ represents H, it is preferable that X denote an integer of 20-24 and Y denote an integer of 9-13; when R₂ represents OH, it is preferable that X denote an integer of 18-24 and Y denote an integer of 9-13; further when R₂ represents H, it is particularly preferable that X denote an integer of 21-23 and Y denote an integer of 10-12; and when R₂ represents OH, it is particularly preferable that X denote an integer of 20-23 and Y denote an integer of 10-12;

(d) —CH═CH(CH₂)_{Y}CH₃,

wherein R₂ represents H and it is preferable that X denote an integer of 10-18 and Y denote an integer of 10-14; and it is particularly preferable that X denote an integer of 11-17 and Y denote an integer of 11-13; and

(e) —CH(OH)(CH₂)_{Y}CH(CH₃)CH₂CH₃,

wherein R₂ represents OH and it is preferable that X denote an integer of 21-25 and Y denote an integer of 9-13; and it is particularly preferable that X denote an integer of 22-24 and Y denote an integer of 10-12.

On the other hand, R₁ in the formula (XXI) preferably represents —CH₂(CH₂)_{Y}CH₃, wherein Y denote preferably an integer of 11-15, particularly 12-14.

A compound of the present invention which has the configurations at 2- and 3-positions as shown in the following formula (II) is particularly preferred.

Furthermore, when the synthesized route described in U.S. Pat. No. 5,936,076 is used, α-galactosylceramide represented by the formula (IV) hereinafter wherein X denote an integer of 8-22 and Y denote an integer of 9-13 is the most preferred from the standpoint of easy availability of the raw material.

The more concrete form and the preferred form of the compound of the present invention represented by the formula (A) (formula (I) and (XXI)) can be defined by the following definitions (1)-(4):

(1) the α-galactosylceramides of the formula (I) represented by the formula (II):

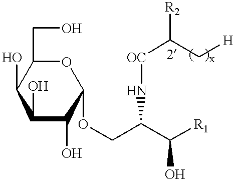

wherein R₁ represents any one of the substituents defined by the following (a)-(e), R₂ represents H or OH and X is defined in the following (a)-(e):

(a) —CH₂(CH₂)_{Y}CH₃,

wherein, when R₂ represents H, X denotes an integer of 0-24 and Y denotes an integer of 7-15; and when R₂ represents OH, X denotes an integer of 20-24 and Y denotes an integer of 11-15;

(b) —CH(OH)(CH₂)_{Y}CH₃,

wherein when R₂ represents H, X denotes an integer of 18-26 and Y denotes an integer of 5-15; and when R₂ represents OH, X denotes an integer of 18-26 and Y denotes an integer of 5-17;

(c) —CH(OH)(CH₂)_{Y}CH(CH₃)₂,

wherein when R₂ represents H, X denotes an integer of 20-24 and Y denotes an integer of 9-13; and when R₂ represents OH, X denotes an integer of 18-24 and Y denotes an integer of 9-13;

(d) —CH═CH(CH₂)_{Y}CH₃,

wherein R₂ represents H, X denotes an integer of 10-18 and Y denotes an integer of 10-14; and

(e) —CH(OH)(CH₂)_{Y}CH(CH₃)CH₂CH₃,

wherein R₂ represents OH, X denotes an integer of 21-25 and Y denotes an integer of 9-13;

(2) the α-galactosylceramides of the formula (1) represented by the formula (III):

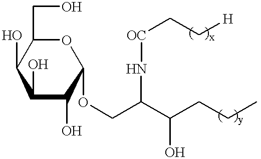

wherein X denotes an integer of 0-24 and Y denotes an integer of 7-15;

(3) the α-galactosylceramides described in the above (2), wherein more preferably X denotes an integer of 8-22 and Y denotes an integer of 9-13;

(4) the α-galactosylceramides described in the above (2) which is more preferably represented by the formula (IV):

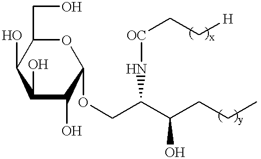

Wherein X denotes an integer of 0-24 and Y denotes an integer of 7-15;

(5) the α-galactosylceramides described in the above (4), wherein most preferably X denotes an integer of 8-22 and Y denotes an integer of 9-13;

(6) the α-galactosylceramides of the formula (I) represented by the formula (V): wherein X denotes an integer of 20-24 and Y denotes an integer of 11-15;

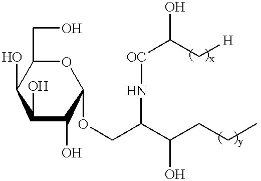

(7) the α-galactosylceramides described in the above (6), wherein more preferably X denotes an integer of 21-23 and Y denotes an integer of 12-14;

(8) the α-galactosylceramides described in the above (6), represented more preferably by the formula (VI):

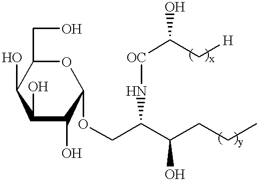

wherein X denotes an integer of 20-24 and Y denotes an integer of 11-15;

(9) the α-galactosylceramides describe in the above (8), wherein more preferably X denotes an integer of 21-23 and Y denotes an integer of 12-14;

(10) the α-galactosylceramides of the formula (I) represented by the formula (VII):

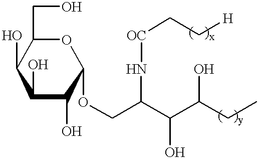

wherein X denotes an integer of 18-26 and Y denotes an integer of 5-15;

(11) the α-galactosylceramides described in the above (10), wherein more preferably X denotes an integer of 21-25 and Y denotes an integer of 6-14;

(12) the α-galactosylceramides described in the above (10) which is represented more preferably by the formula (VIII):

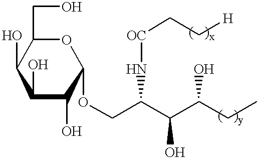

wherein X denotes an integer of 18-26 and Y denotes an integer of 5-15;

(13) the α-galactosylceramides described in the above (12), wherein most preferably X denotes an integer of21-25 and Y denotes an integer of 6-14;

(14) the α-galactosylceramides of the formula (I) represented by the formula (IX):

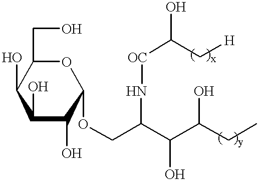

wherein X denotes an integer of 18-26 and Y denotes an integer of 5-17;

(15) the α-galactosylceramides described in the above (14), wherein more preferably X denotes an integer of 21-25 and Y denotes an integer of 6-16;

(16) the α-described in the above (10) which is represented more preferably by the formula (X):

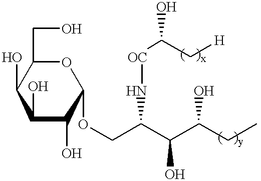

wherein X denotes an integer of 18-26 and Y denotes an integer of 5-17;

(17) the α-galactosylceramides described in the above (14) which is represented more preferably by the formula (X′):

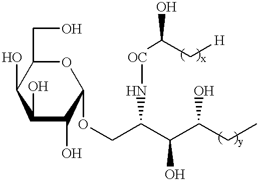

wherein X denotes an integer of20-24 and Y denotes an integer of 10-4;

(18) the α-galactosylceramides described in the above (16), wherein more preferably X denotes an integer of 21-25 and Y denotes an integer of 6-16;

(19) the α-galactosylceramides described in the above (17), wherein most preferably X denotes an integer of 21-23 and Y denotes an integer of 11-13;

(20) the α-galactosylceramides of the formula (I) represented by the formula (XI):

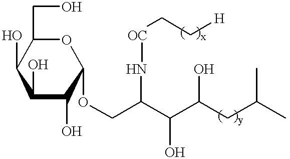

wherein X denotes an integer of 20-24 and Y denotes an integer of 9-13;

(21) the α-galactosylceramides described in the above (20), wherein more preferably X denotes an integer of 21-23 and Y denotes an integer of 10-12;

(22) the α-galactosylceramides described in the above (20) more preferably represented by the formula (XII):

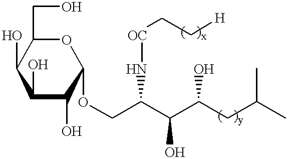

wherein X denotes an integer of 20-24 and Y denotes an integer of 9-13;

(23) the α-galactosylceramides described in the above (22), wherein more preferably X denotes an integer of 21-23 and Y denotes an integer of 10-12;

(24) the α-galactosylceramides of the formula (I) represented by the formula (XIII):

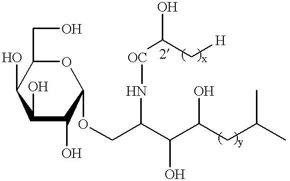

wherein X denotes an integer of 18-24 and Y denotes an integer of 9-13;

(25) the α-galactosylceramides described in the above (24), wherein more preferably X denotes an integer of 20-23 and Y denotes an integer of 10-12;

(26) the α-galactosylceramides described in the above (24), more preferably represented by the formula (XIV):

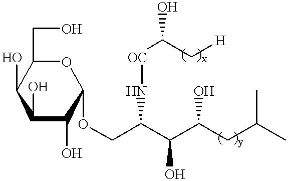

wherein X denotes an integer of 19-23 and Y denotes an integer of 9-13;

(27) the α-galactosylceramides described in the above (24), more preferably represented by the formula (XIV):

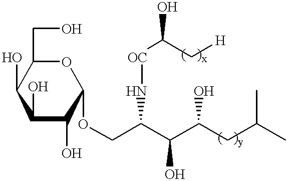

wherein X denotes an integer of 20-24 and Y denotes an integer of 9-14;

(28) the α-galactosylceramides described in the above (26), wherein most preferably X denotes an integer of 20-22 and Y denotes an integer of 10-12;

(29) the α-galactosylceramides described in the above (27), wherein most preferably X denotes an integer of 21-23 and Y denotes an integer of 10-12;

(30) the α-galactosylceramides of the formula (I) represented by the formula (XV):

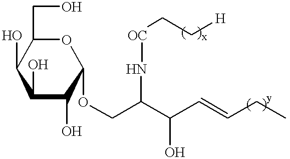

wherein X denotes an integer of 10-18 and Y denotes an integer of 10-14;

(31) the α-galactosylceramides described in the above (30), wherein more preferably X denotes an integer of 11-17 and Y denotes an integer of 11 -1 3;

(32) the α-galactosylceramides described in the above (30) more preferably represented by the formula (XVI):

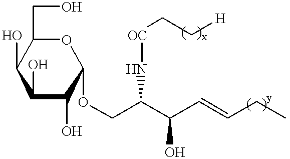

wherein X denotes an integer of 10-18 and Y denotes an integer of 10-14;

(33) The α-galactosylceramides described in the above (32), wherein most preferably X denotes an integer of 11-17 and Y denotes an integer of 11-13;

(34) the α-galactosylceramides of the formula (I) represented by the formula (XVII):

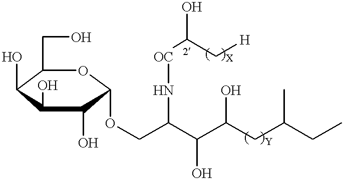

wherein X denotes an integer of 21-25 and y denotes an integer of 9-13;

(35) the α-galactosylceramides described in the above (34), wherein more preferably X denotes an integer of 22-24 and Y denotes an integer of 22-24 and Y denotes an integer of 10-12;

(36) the α-galactosylceramides described in the above (34) more preferably represented by the formula (XVIII):

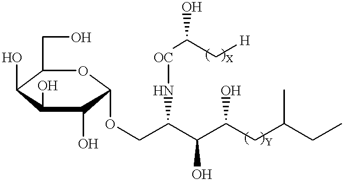

wherein X denotes an integer of 21-25 and Y denotes an integer of 9-13;

(37) the α-galactosylceramides described in the above (36), wherein most preferably X denotes an integer of 22-24 and Y denotes an integer of 10-12;

(38) the α-galactosylceramides of the formula (XXI) represented by the formula (XIX):

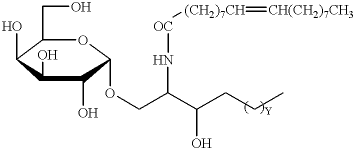

wherein Y denotes an integer of 11-15;

(39) the α-galactosylceramides described in the above (38), wherein most preferably Y denotes an integer of 12-14;

(40) the α-galactosylceramide described in the above (38) more preferably represented by the formula (XX):

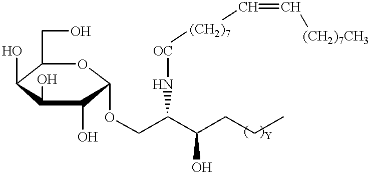

wherein Y denotes an integer of 11-15; and

(41) the α-galactosylceramides described in the above (40), wherein most preferably Y denotes an integer of 12-14.

Concrete preferred examples of compounds included in the present invention represented by the formula (A) (formula (I) and (XXI)) are shown below. In respective formula, X and Y are defined as above.

(1) The compounds represented by the following formula (III) and (VI)

TABLE 2
(1) The compounds represented by the following formula (III) and (VI)

| | |
|---|---|
| | (III) |
| 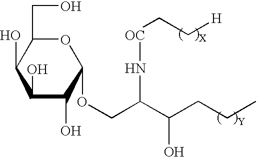 | |
| | (VI) |
| 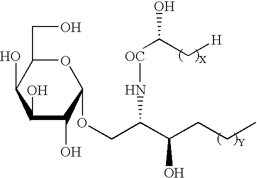 | |

Compound 1:
(2S,3R)-1-(α-D-galactopyranosyloxy)-2-tetracosanoylamino-3-octadecanol,
Compound 2:
(2S,3R)-2-docosanoylamino-1-(α-D-galactopyranosyloxy)-3-octadecanol,
Compound 3:
(2S,3R)-1-(α-D-galactopyranosyloxy)-2-eicosanoylamino(icosanoylamino)-3-
octadeconol,
Compound 4:
(2S,3R)-1-(α-D-galactopyranosyloxy)-2-octadecanoylamino-3-octadeconol,
Compound 5:
(2S,3R)-1-(α-D-galactopyranosyloxy)-2-tetradecanoylamino-3-octadecanol,
Compound 6:
(2S,3R)-2-decanoylamino-1-(α-D-galactopyranosyloxy)-3-octadecanol,
Compound 7:
(2S,3R)-1-(α-D-galactopyranosyloxy)-2-octanoylamino-3-octadecanol,
Compound 8:
(2S,3R)-2-acetamino-1-(α-D-galactopyranosyloxy)-(3-octadecanol,
Compound 9:
(2S,3R)-1-(α-D-galactopyranosyloxy)-2-tetracosanoylamino-3-tetradecanol,
Compound 10:
(2S,3R)-1-(α-D-galactopyranosyloxy)-2-tetradecanoylamino-3-hexadecanol,
Compound 11:
(2R,3S)-1-(α-D-galactopyranosyloxy)-2-tetradecanoylamino-3-hexadecanol,
Compound 12:
(2S,3S)-1-(α-D-galactopyranosyloxy)-2-tetradecanoylamino-3-hexadecanol,
Compound 13:
(2R,3R)-1-(α-D-galactopyranosyloxy)-2-tetradecanoylamino-3-hexadecanol,
Compound 14:
(2S,3R)-1-(α-D-galactopyranosyloxy)-2-[(R)-2-hydroxytetracosanoylamino]-3-
octadecanol.
Among these compounds, the compounds 1-10 and 14 are preferred in consideration
of the configuration at 2- and 3-positions.
(2) The compounds represented by the following formula (XVI)

| | |
|---|---|
| | (XVI) |
| 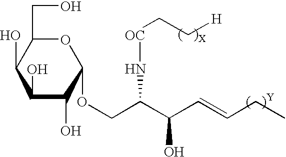 | |

Compound 15:
(2S,3R,4E)-1-(α-D-galactopyranosyloxy)-2-octadecanoylamino-4-octadecen-3-ol,
Compound 35:
(2S,3R,4E)-1-(α-D-galactopyranosyloxy)-2-tetradecanoylamino-4-octadecen-3-
ol.
(3) The compounds represented by the following formula (VIII)

| | |
|---|---|
| | (VIII) |
| 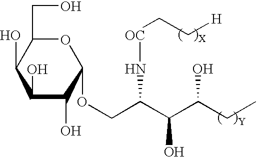 | |

Compound 16:
(2S,3S,4R)-1-(α-D-galactopyranosyloxy)-2-tetracosanoylamino-3,4-
octadecanediol,
Compound 17:
(2S,3S,4R)-1-(α-D-galactopyranosyloxy)-2-tetracosanoylamino-3,4-
hepadecanediol,
Compound 18:
(2S,3S,4R)-1-(α-D-galactopyranosyloxy)-2-tetracosanoylamino-3,4-
pentadecanediol,
Compound 19:
(2S,3S,4R)-1-(α-D-galactopyranosyloxy)-2-tetracosanoylamino-3,4-
undecanediol,
Compound 20:
(2S,3S,4R)-1-(α-D-galactopyranosyloxy)-2-hexacosanoylamino-3,4-
heptadecanediol,
Compound 36:
(2S,3S,4R)-1-(α-D-galactopyranosyloxy)-2-hexacosanoylamino-3,4-octadecanediol,
Compound 37:
(2S,3S,4R)-1-(α-D-galactopyranosyloxy)-2-octacosanoylamino-3,4-heptadecanediol.
(4) The compounds represented by the following formula (X) and (X′)

| | |
|---|---|
| | (X) |
| 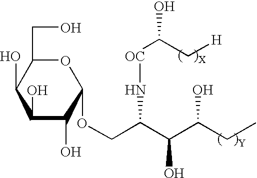 | |
| | (X′) |
| 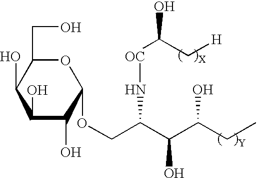 | |

Compound 23:
(2S,3S,4R)-1-(α-D-galactopyranosyloxy)-2-[(R)-2-hydroxytetracosanoylamino]-
3,4-pentadecanediol,
Compound 24:
(2S,3S,4R)-1-(α-D-galactopyranosyloxy)-2-[(R)-2-hydroxytetracosanoylamino]-3,4-
undecanediol,
Compound 25:
(2S,3S,4R)-1-(α-D-galactopyranosyloxy)-2-[(R)-2-hydroxytetracosanoylamino]-
3,4-octadecanediol,
Compound 26:
(2S,3S,4R)-1-(α-D-galactopyranosyloxy)-2-[(R)-2-hydroxyhexacosanoylamino]-
3,4-nonadecanediol,
Compound 27:
(2S,3S,4R)-1-(α-D-galactopyranosyloxy)-2-[(R)-2-hydroxytetracosanoylamino]-
3,4-eicosanediol (icosanediol),
Compound 28:
(2S,3S,4R)-1-(α-D-galactopyranosyloxy)-2-[(S)-2-hydroxytetracosanoylamino]-
3,4-heptadecanediol,
Compound 32:
(2S,3S,4R)-1-(α-D-galactopyranosyloxy)-2-[(R)-2-hydroxytetracosanoylamino]-
3,4-hexadecanediol.
(5) The compounds represented by the following formula (XII), (XIV) and (XIV′)

| | |
|---|---|
| | (XII) |
| 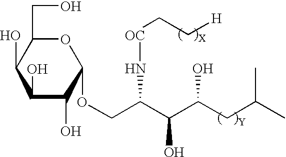 | |
| | (XIV) |
| 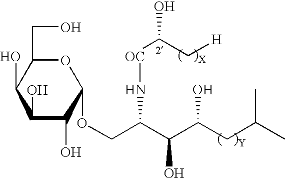 | |
| | (XIV′) |
| 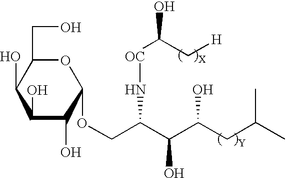 | |

Compound 30:
(2S,3S,4R)-1-(α-D-galactopyranosyloxy)-2-[(S)-2-hydroxytetracosanoylamino]-16-
methyl-3,4-heptadecanediol,
Compound 31:
(2S,3S,4R)-1-(α-D-galactopyranosyloxy)-16-methyl-2-tetracosanoylamino]-3,4-
heptadecanediol,
Compound 33:
(2S,3S,4R)-1-(α-D-galactopyranosyloxy)-2-[(R)-2-hydroxytetracosanoylamino]-16-
methyl-3,4-heptadecanediol,
(6) The compound represented by the following formula (XVIII)

| | |
|---|---|
| | (XIX) |
| 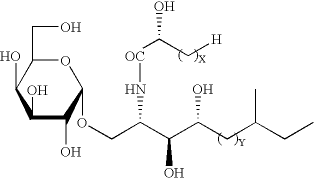 | |

Compound 34:
(2S,3S,4R)-1-(α-D-galactopyranosyloxy)-2-[(R)-2-hydroxytetracosanoylamino]-16-
methyl-3,4-octadecanediol.
(7) The compound represented by the following formula (XIX)

| | |
|---|---|
| | (XIX) |
| 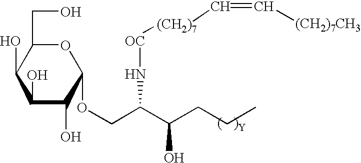 | |

Compound 29:
(2S,3R)-1-(α-D-galactopyranosyloxy)-2-oleoylamino]-3-octadecanol.

All references, patents and patent publications that are recited in this application are incorporated in their entirety herein by reference.

What is claimed is:

1. A method for treating an infectious disease in a subject in need thereof, comprising:
administering to the subject, an alpha-glycosylceramide in an amount effective to treat the infectious disease in the subject,
wherein the alpha-glycosylceramide is selected from the group consisting of an alpha-galactosylceramide and an alpha-glucosylceramide,
wherein the subject is not otherwise in need of administration of an alpha-galactosylceramide or an alpha-glucosylceramide, and
wherein the infectious disease is a bacterial infectious disease or a fungal infectious disease.

2. The method of claim 1, wherein the infectious disease is a bacterial infectious disease.

3. The method of claim 1, wherein the infectious disease is a fungal infectious disease.

4. The method of claim 1, wherein the subject has a bacterial infectious disease selected from the group consisting of Helicobacter pyloris, Borelia burgdorferi, Legionella pneumophilia, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus antracis, corynebacterium diphtheriae, corynebacterium sp., Erysipelothrix rhusiopathiae, Clostridium perfringers, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, Rickettsia, Actinomyces israelli, and Salmonella spp.

5. The method of claim 1, wherein the subject has a fungal infectious disease selected from the group consisting of: Cryptococcus neoformans, Histoplasma capsulatum, Coccidioides immitis, Blastomyces dermatitidis, Chlamydia trachomatis, and Candida albicans.

6. The method of claim 1, wherein the alpha-glycosylceramide is an alpha-galactosylceramide.

7. The method of claim 1, wherein the alpha-glycosylceramide is an alpha-glucosylceramide.

8. The method of claim 1, wherein the alpha-glycosylceramide is an alpha-galactosylceramide.

9. The method of claim 1, wherein the alpha-glycosylceramide is an alpha-glucosylceramide.

10. The method of claim 1, wherein administering comprises orally administering the alpha-glycosylceramide to the subject.

11. The method of claim 1, wherein administering comprises aerosol administration.

12. The method of claim 1, wherein administering comprises co-administering an anti-infective agent to the subject.

13. The method of claim 1, wherein the infectious disease is a bacterial infectious disease, further comprising the step of administering an antibacterial agent to the subject.

14. The method of claim 1, wherein the infectious disease is a fungal infectious disease, further comprising the step of administering an antifungal agent to the subject.

15. A pharmaceutical composition comprising:
an alpha-glycosylceramide;
an anti-infective agent that is an anti-bacterial agent or an anti-fungal agent; and
a pharmaceutically acceptable carrier.

16. The composition of claim 15, wherein the alpha-glycosylceramide is an alpha-galactosylceramide.

17. The composition of claim 15, wherein the alpha-glycosylceramide is an alpha-glucosylceramide.

18. The composition of claim 15, wherein the anti-infective agent is an anti-bacterial agent.

19. The composition of claim 15, wherein the anti-infective agent is an anti-fungal agent.

20. A screening method to identify putative alpha-glycosylceramide molecules that can stimulate NKT cells through a CD1d mechanism, comprising:
performing an NKT stimulation assay in the presence and absence of a putative alpha-glycosylceramide molecule; and
detecting a shift toward a Th1 response,
wherein a shift toward a Th1 response in the presence of the putative alpha-glycosylceramide molecule indicates that the putative agent is an alpha-glycosylceramide as used herein.
